SEQUENCE LISTING (50 sequences)

SEQ ID NO: 1 - RNA, 21 nt, Mus musculus
uggaauguaaagaaguaugua

SEQ ID NO: 2 - RNA, 21 nt, Mus musculus
uaaagcgcuuuuuacauuccu

SEQ ID NO: 3 - RNA, 22 nt, Homo sapiens
uggaauguaaagaaguauguaa

SEQ ID NO: 4 - RNA, 23 nt, Homo sapiens
uaaaagcgcuuuuuuacauuccu

SEQ ID NO: 5 - RNA, 21 nt, Mus musculus
uguguacuccucuacauucca

SEQ ID NO: 6 - RNA, 23 nt, Mus musculus
aaaagagaccuuucacauucaug

SEQ ID NO: 7 - RNA, 18 nt, Mus musculus
ugccacuucuucauucug

SEQ ID NO: 8 - RNA, 25 nt, Homo sapiens
ucaacagugccacuucugcauucuc

SEQ ID NO: 9 - RNA, 21 nt, Mus musculus
cacaucuucacuacauuacca

SEQ ID NO: 10 - RNA, 23 nt, Homo sapiens
cacaucaucguuuuccauuugca

SEQ ID NO: 11 - RNA, 25 nt, Mus musculus
aacaauacuucuuugaccgugaccc

SEQ ID NO: 12 - RNA, 27 nt, Homo sapiens
ucagcaguauuucuuugaccgugaccc

SEQ ID NO: 13 - RNA, 27 nt, Artificial Sequence
  Synthetic nucleic acid
ucagcaguauuaaauugaccgugaccc

SEQ ID NO: 14 - RNA, 36 nt, Artificial Sequence
  Synthetic nucleic acid
guccgggucacgguccccaaagaaguauuguugggu

SEQ ID NO: 15 - RNA, 30 nt, Artificial Sequence
  Synthetic nucleic acid
uauugggaaucaaaagaugguaaauguucc

SEQ ID NO: 16 - RNA, 31 nt, Artificial Sequence
  Synthetic nucleic acid
ggcuuggaaugaacaaaacuuuucauuugac

SEQ ID NO: 17 - RNA, 22 nt, Mus musculus
agccuuacuucuuuucucucug

SEQ ID NO: 18 - RNA, 26 nt, Homo sapiens
uagaaaauacuucuuuauucauguuu

SEQ ID NO: 19 - RNA, 22 nt, Artificial Sequence
  Synthetic nucleic acid
agccuuacuucuuuucucucug

SEQ ID NO: 20 - RNA, 22 nt, Artificial Sequence
  Synthetic nucleic acid
agccuuaaaacuuuucucucug

SEQ ID NO: 21 - RNA, 22 nt, Mus musculus
cauauccuucuuugcacuccca

SEQ ID NO: 22 - RNA, 21 nt, Homo sapiens
aucuccuucuuugcgcuccca

SEQ ID NO: 23 - RNA, 29 nt, Artificial Sequence
  Synthetic nucleic acid
gcugggagugcaaagaaggauauggcgaa

SEQ ID NO: 24 - RNA, 22 nt, Mus musculus
ugcuuuccuucuuuccauucca

SEQ ID NO: 25 - RNA, 20 nt, Homo sapiens
ugauuacuucuuauuuuucu

SEQ ID NO: 26 - RNA, 20 nt, Mus musculus
uucacucuucuuuauggcug

SEQ ID NO: 27 - RNA, 19 nt, Mus musculus
ugccgccuucuuuugaccu

SEQ ID NO: 28 - RNA, 21 nt, Homo sapiens
uucgacuucuuggcagaguug

SEQ ID NO: 29 - RNA, 20 nt, Mus musculus
aaugacuuuucaacauuuca

SEQ ID NO: 30 - RNA, 21 nt, Homo sapiens
uaaugacauuucaacauuuca

SEQ ID NO: 31 - RNA, 22 nt, Mus musculus
uacaucuucuauuuacucuuug

SEQ ID NO: 32 - RNA, 21 nt, Mus musculus
uaauuguuucuuuggggaaag

SEQ ID NO: 33 - RNA, 21 nt, Mus musculus
uaucauuuucuucacgucucu

SEQ ID NO: 34 - RNA, 22 nt, Mus musculus
uacuguaggaucuuacauucug

SEQ ID NO: 35 - RNA, 22 nt, Mus musculus
cauuuaccaucuuuugauuccc

SEQ ID NO: 36 - RNA, 21 nt, Mus musculus
ugaaaaguuuuguucauucca

SEQ ID NO: 37 - RNA, 24 nt, Mus musculus
auacaggcuucuuuacauaaacca

SEQ ID NO: 38 - RNA, 26 nt, Homo sapiens
uagaaaauacuucuuuauucauguuu

SEQ ID NO: 39 - RNA, 30 nt, Artificial Sequence
  Synthetic nucleic acid
uccugguuuauguaaagaagccuguauuuc

SEQ ID NO: 40 - RNA, 21 nt, Mus musculus
uccacuucuucgucauuauca

SEQ ID NO: 41 - RNA, 23 nt, Homo sapiens
uccauacuucuuuauuauugaua

SEQ ID NO: 42 - RNA, 31 nt, Artificial Sequence
  Synthetic nucleic acid
guugcugauaaugacgaagaaguggacccac

SEQ ID NO: 43 - RNA, 21 nt, Mus musculus
uagcagcacagaaauauuggc

SEQ ID NO: 44 - RNA, 22 nt, Mus musculus
gcucuauguuucuggagagcua

SEQ ID NO: 45 - RNA, 23 nt, Homo sapiens
gcucuauguuucuggagagcuca

SEQ ID NO: 46 - RNA, 23 nt, Mus musculus
gugguucauuucugggcaccaug

SEQ ID NO: 47 - RNA, 21 nt, Homo sapiens
uagcagcacagaaauauuggc

SEQ ID NO: 48 - RNA, 21 nt, Homo sapiens
gguccauuucugggcaccaug

SEQ ID NO: 49 - RNA, 20 nt, Homo sapiens
cccacuuuucuguuuuucaa

SEQ ID NO: 50 - RNA, 29 nt, Artificial Sequence
  Synthetic nucleic acid
cuauuagcucuccagaaacauagagcuga

(19) United States
(12) Patent Application Publication
(10) Pub. No.: US 2010/0029501 A1
(43) Pub. Date: Feb. 4, 2010

(54) METHOD OF IDENTIFYING MICRO-RNA TARGETS

(76) Inventors: Eva Samal, San Francisco, CA (US); Deepak Srivastava, Orinda, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/496,481

(22) Filed: Jul. 1, 2009

Related U.S. Application Data
(60) Provisional application No. 61/134,518, filed on Jul. 9, 2008.

(51) Int. Cl. C40B 30/04; G01N 33/566; C12Q 1/68

(52) U.S. Cl. 506/9; 436/501; 435/6

(57) ABSTRACT

The present invention provides methods of identifying an mRNA target of a microRNA. The present invention further provides kits and systems for carrying out a subject method.

### CROSS-REFERENCE

This application claims the benefit of U.S. Provisional Patent Application No. 61/134,518, filed Jul. 9, 2008, which application is incorporated herein by reference in its entirety.

### STATEMENT REGARDING FEDERALLY SPONSORED RESEARCH

The U.S. government has certain rights in this invention, pursuant to grant no. C06 RR018928 awarded by the National Institutes of Health.

### BACKGROUND

Animal microRNAs (miRNAs) are genomically encoded 20-26 nucleotide (nt) RNA molecules that function predominantly as post-transcriptional negative regulators by inhibiting translation or mediating target mRNA degradation. Over 450 human miRNAs have been identified that regulate diverse processes, including cell cycle and differentiation, development, and tissue homeostasis. miRNAs have also been linked to human diseases, e.g., cancer. However, few targets of miRNAs have been validated, largely because of the lack of perfect complementarity required for miRNA:mRNA association and the limited knowledge of the “rules” of interaction.

Most potential targets of miRNAs have been determined computationally by the presence of an uninterrupted sequence match with bases 1-7 or 2-8 at the 5′ end of the miRNA, commonly known as the “seed” sequence. To decrease the promiscuity of in silico predictions based solely on seed matches, computer algorithms often use phylogenetic sequence conservation of a potential miRNA-interacting site. Since the secondary structure of the mRNA target region may affect the accessibility to its binding site, free energy (ΔG) calculations of the mRNA binding site region have been used in an attempt to increase specificity. While such bioinformatics approaches have been helpful, various computational predictions generate large and diverse outputs with limited overlap between target data sets even for the same miRNA. Few computationally predicted targets have been validated in vivo, highlighting the need for direct experimental methods to identify miRNA targets.

There is a need in the art for methods of identifying mRNA targets of miRNAs.

### Literature

Long et al. (2007) Nat. Struct. Mol. Biol. 14:287; Stefani and Slack (2006) Cold Spring Harbor Symp. Quant. Biol. 71:129; Zhao et al. (2007) Cell 129:303; Zhao et al. (2005) Nature 436:214; U.S. Patent Publication No. 2007/0092882; U.S. Patent Publication No. 2004/0175732.

### SUMMARY OF THE INVENTION

The present invention provides methods of identifying an mRNA target of a microRNA. The present invention further provides kits and systems for carrying out a subject method.

### BRIEF DESCRIPTION OF THE DRAWINGS

FIGS. 1A-E depict a biochemical screen for miR-1 targets.

FIGS. 2A-I depict experimental validation of an miR-1 target screen.

FIGS. 3A-I depict validation of miR-1 targets with non-canonical 5′ seeds.

FIGS. 4A-D depict validation of enriched miR-1 targets affecting cell cycle.

FIGS. 5A-H depict evidence for an alternative seed sequence for miRNA-mediated repression.

FIGS. 6A-H depict validation of the alternate see sequence for miR-1-mediated repression on various targets.

FIGS. 7A-D depicts the effect of miR-1-2 overexpression on cardiac physiology.

FIGS. 8A-C depict validation of pull-down and in vivo mouse model.

FIGS. 9A-C depict statistics for miR-1 pull-down.

FIGS. 10A-D depict putative miR-1 binding sites in miR-1 pull-down enriched targets.

FIGS. 11A-J depict data relating to the seed region in miR-1-mediated repression.

FIG. 12 is a table that provides a list of annotated mRNAs enriched ≧8-fold in a miR-1 pulldown assay.

FIGS. 13A-E depict repression mediated by the middle region of miR-195.

### DEFINITIONS

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome which are capable of modulating the productive utilization of mRNA. An artificial microRNA can be any type of RNA sequence, other than endogenous microRNA, which is capable of modulating the activity of an mRNA. A microRNA sequence can be an RNA molecule composed of any one or more of these sequences. MicroRNA sequences have been described in publications such as, Lim, et al., 2003, Genes & Development, 17, 991-1008, Lim et al., 2003, Science, 299, 1540, Lee and Ambrose, 2001, Science, 294, 862, Lau et al., 2001, Science 294, 858-861, Lagos-Quintana et al., 2002, Current Biology, 12, 735-739, Lagos-Quintana et al., 2001, Science, 294, 853-857, and Lagos-Quintana et al., 2003, RNA, 9, 175-179, which are incorporated herein by reference. Examples of microRNAs include any RNA that is a fragment of a larger RNA or is a miRNA, siRNA, stRNA, sncRNA, tncRNA, snoRNA, smRNA, snRNA, or other small non-coding RNA. See, e.g., US Patent Publication Nos. 20050272923, 20050266552, 20050142581, and 20050075492. A “microRNA precursor” refers to a nucleic acid having a stem-loop structure with a microRNA sequence incorporated therein.

A “stem-loop structure” refers to a nucleic acid having a secondary structure that includes a region of nucleotides which are known or predicted to form a double strand (step portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches.

The term “biological sample” encompasses a variety of sample types obtained from an organism and can be used in subject method. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components such as mRNA. The term encompasses a clinical sample, and also includes cells in cell culture, cell supernatants, cell lysates, biological fluids, and tissue samples.

A “substantially isolated” or “isolated” mRNA is one that is substantially free of the materials with which it is associated in nature. By substantially free is meant at least 50%, at least 70%, at least 80%, or at least 90% free of the materials with which it is associated in nature. In some embodiments, an isolated mRNA is purified, e.g., the mRNA is at least about 70%, at least about 80%, at least about 90%, at least about 95%, or at least about 99%, or more, pure (e.g., free of non-mRNA macromolecules, small molecule contaminants, etc.).

“Probe,” as used herein, is defined as a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G. U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages.

An “array” may comprise a solid support with peptide or nucleic acid probes attached to the support. Arrays typically comprise a plurality of different nucleic acid probes that are coupled to a surface of a substrate in different, known locations. These arrays, also referred to as “microarrays” or colloquially “chips,” have been generally described in the art, for example, U.S. Pat. Nos. 5,143,854, 5,445,934, 5,744,305, 5,677,195, 6,040,193, 5,424,186 and Fodor et al., Science, 251:767 777 (1991). These arrays can generally be produced using mechanical synthesis methods or light directed synthesis methods which incorporate a combination of photolithographic methods and solid phase synthesis methods. Techniques for the synthesis of these arrays using mechanical synthesis methods, such as ink jet, channel block, flow channel, and spotting methods which are described in, e.g., U.S. Pat. Nos. 5,384,261, and 6,040,193. Although a planar array surface is preferred, the array may be fabricated on a surface of virtually any shape or even a multiplicity of surfaces. Arrays can be nucleic acids on beads, gels, polymeric surfaces, fibers such as fiber optics, glass or any other appropriate substrate, see U.S. Pat. Nos. 5,744,305, 5,770,358, 5,789,162, 5,708,153, 6,040,193 and 5,800,992. Arrays may be packaged in such a manner as to allow for diagnostics or other manipulation of in an all-inclusive device, see for example, U.S. Pat. Nos. 5,856,174 and 5,922,591, and 5,945,334.

Nucleic acid hybridization reactions can be performed under conditions of different “stringency”. Conditions that increase stringency of a hybridization reaction of widely known and published in the art. See, e.g., Sambrook et al. Molecular Cloning, A Laboratory Manual, 2nd Ed., Cold Spring Harbor Laboratory Press, Cold Spring Harbor, N.Y., 1989, herein incorporated by reference. For example, see page 7.52 of Sambrook et al. Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where 1×SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1×SSC (150 mM NaCl, 15 mM sodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. As another example, stringent hybridization conditions comprise: prehybridization for 8 hours to overnight at 65° C. in a solution comprising 6× single strength citrate (SSC) (1× SSC is 0.15 M NaCl, 0.015 M Na citrate; pH 7.0), 5× Denhardt's solution, 0.05% sodium pyrophosphate and 100 μg/ml herring sperm DNA; hybridization for 18-20 hours at 65° C. in a solution containing 6× SSC, 1× Denhardt's solution, 100 μg/ml yeast tRNA and 0.05% sodium pyrophosphate; and washing of filters at 65° C. for 1 hour in a solution containing 0.2× SSC and 0.1% SDS (sodium dodecyl sulfate).

Stringent hybridization conditions are hybridization conditions that are at least as stringent as the above representative conditions. Other stringent hybridization conditions are known in the art and may also be employed to identify nucleic acids of this particular embodiment of the invention.

A polynucleotide has a certain percent “sequence identity” to another polynucleotide, meaning that, when aligned, that percentage of bases are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments, with a restricted affine gap penalty model. See Meth. Mol. Biol. 70: 173-187 (1997). Also, the GAP program using the Needleman and Wunsch alignment method can be utilized to align sequences using a general class of gap models. See J. Mol. Biol. 48: 443-453 (1970).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a microRNA” includes a plurality of such microRNAs and reference to “the mRNA” includes reference to one or more mRNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides methods of identifying an mRNA target of a microRNA (miRNA). The methods generally involve contacting the miRNA with a plurality of mRNAs under conditions that favor duplex formation between the miRNA and at least one member of the plurality of mRNAs; and eluting any mRNA that forms a duplex with the miRNA. The eluted mRNA can then be analyzed using any of a variety of methods. The present invention further provides kits and systems for carrying out a subject method.

Methods of Identifying an mRNA Target of a microRNA

In general, a subject method of identifying an mRNA target of a miRNA involves contacting a miRNA with a plurality of mRNAs under conditions that favor binding of at least one member (“species”) of the plurality of mRNAs to the miRNA, forming an miRNA/mRNA complex or duplex; and eluting any bound mRNA from the duplex.

miRNA

The miRNA generally has a known sequence. A variety of miRNAs are known, and the nucleotide sequences of many miRNAs are publicly available. See, e.g., Lagos-Quintana et al. (2001) Science 294:853; Landgraf et al. (2007) Cell 129:1401; and on the internet at microrna(dot)sanger(dot)ac(dot)uk/. Any known miRNA can be used. In addition, a miRNA comprising a sequence not present in publicly available databases can be used.

The miRNA can be isolated from a biological source, or can be synthesized (e.g., synthesized in a laboratory in a cell-free in vitro system, including, e.g., via chemical synthesis). For example, a miRNA can be chemically synthesized, where nucleic acid synthesis is performed according to standard methods. See, for example, Itakura and Riggs (1980). Additionally, U.S. Pat. No. 4,704,362, U.S. Pat. No. 5,221,619, and U.S. Pat. No. 5,583,013 each describes various methods of preparing synthetic nucleic acids. Non-limiting examples of a synthetic nucleic acid (e.g., a synthetic oligonucleotide), include a nucleic acid made by in vitro chemical synthesis using phosphotriester, phosphite, or phosphoramidite chemistry and solid phase techniques such as described in EP 266,032, or via deoxynucleoside H-phosphonate intermediates as described in U.S. Pat. No. 5,705,629. Various methods of oligonucleotide synthesis have been disclosed, and can be used to synthesize a miRNA; see, e.g., U.S. Pat. Nos. 4,659,774, 4,816,571, 5,141,813, 5,264,566, 4,959,463, 5,428,148, 5,554,744, 5,574,146, 5,602,244.

The miRNA can comprise a nucleotide sequence found in an endogenous miRNA, e.g, the sequence is a naturally-occurring sequence. Alternatively, the miRNA can comprise a nucleotide not found in an endogenous miRNA, e.g., where the miRNA comprises a non-naturally occurring sequence.

Non-limiting examples of miRNAs that can be used include those in Table 1 of U.S. Patent Publication No. 2007/0092882; and in Table 1 of U.S. Patent Publication No. 2008/0026951.

The miRNA can include a mature miRNA sequence, and can have a length of from about 19 nt to about 21 nt, from about 21 nt to about 23 nt, from about 23 nt to about 25 nt, from about 25 nt to about 27 nt, from about 27 nt to about 30 nt, from about 30 nt to about 35 nt, from about 35 nt to about 40 nt, from about 40 nt to about 50 nt, from about 50 nt to about 75 nt, or from about 75 nt to about 100 nt, or longer than 100 nt, where the mature mRNA sequence (e.g., a mature miRNA sequence having a length of from about 19 nt to about 21 nt, from about 21 nt to about 23 nt, from about 23 nt to about 25 nt, or from about 25 nt to about 27 nt) can be flanked on the 5′ and/or 3′ end by one or more additional nucleotides, resulting in a total length that is longer than the mature miRNA length.

The miRNA can be immobilized on a solid support but need not be. In some embodiments, the miRNA is not immobilized on a solid support and instead is soluble. In some embodiments, the miRNA comprises an amine group on the 3′ end of the miRNA. In some embodiments, the miRNA comprises a biotin moiety covalently linked to the miRNA via an amine group on the 3′ end of the miRNA. In some embodiments, a biotin moiety is conjugated to an miRNA molecule via an esterification reaction.

As an example (e.g., where the miRNA is in solution (not immobilized on a solid support), where the miRNA comprises a 3′ amine and a biotin group attached to the 3′ amine), a plurality of mRNA is contacted with the miRNA under conditions such that at least one species of mRNA in the plurality of mRNA formed a complex with the biotinylated miRNA; and streptavidin immobilized on a solid support (e.g., streptavidin-conjugated magnetic beads) is used to separate miRNA/mRNA complexes from non-complexed mRNA.

In some embodiments, the miRNA is immobilized on a solid support. Suitable solid supports can be of any of a variety of materials and in any of a variety of forms. The insoluble supports may be any compositions to which a nucleic acid (or a nucleic acid modified with a polypeptide) can be bound, which is readily separated from soluble material, and which is otherwise compatible with the overall method. The surface of such supports may be solid or porous and of any convenient shape. Suitable insoluble supports include, e.g., beads (including, e.g., magnetic beads); multiwell plates; and the like. Suitable insoluble supports include, but are not limited to, agarose (e.g., agarose beads), sepharose, glass, plastic (e.g., any of a group of synthetic or natural organic materials that may be shaped when soft and then hardened, including many types of resins, resinoids, polymers, cellulose derivatives, casein materials, and proteins), polypropylene, polystyrene, polystyrene beads, magnetic particles, other microparticles, polystyrene multiwell plates, polypropylene multiwell plates, polycarbonate multiwell plates, and the like. Insoluble supports can take any of a variety of forms, including, but not limited to, beads (which can be spherical, roughly spherical, or irregular in shape), plates, columns, and the like. Plates include multi-well plates (e.g., polystyrene or polypropylene plates) such as multi-well 96-well plates, 384-well plates, 1536-well plates, and the like. Suitable materials which an insoluble support can comprise include glass (e.g., silicon dioxide), plastic (e.g. polystyrene; polypropylene; polycarbonate; etc.), polysaccharides, nylon, and nitrocellulose. A miRNA can be linked to an insoluble support directly or via a linker such as a polypeptide, a member of a specific binding pair (e.g., biotin; an antibody; and the like); etc.

Linkage of a miRNA to an insoluble support can be carried out using any of a variety of chemistries that are well known to those skilled in the art. For example, a miRNA can be modified to include an amine group, where the amine group serves as an attachment moiety for covalent linkage to a moiety that is attached to an insoluble support.

There are several methods and compositions known for derivatizing oligonucleotides with reactive functionalities which permit linkage to a moiety such a biotin, a polypeptide, and the like. For example, several approaches are available for biotinylating nucleic acids such that the biotinylated nucleic acid can be immobilized on an insoluble support via avidin (e.g., where an insoluble support comprises avidin linked thereto). See, e.g., Broken et al., Nucl. Acids Res. (1978) 5:363-384 which discloses the use of ferritin-avidin-biotin labels; and Chollet et al. Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily linked to other compounds that are derivatized by amino-reactive groups, such as isothiocyanate, N-hydroxysuccinimide, or the like, see, e.g., Connolly (1987) Nucl. Acids Res. 15:3131-3139, Gibson et al. (1987) Nucl. Acids Res. 15:6455-6467 and U.S. Pat. No. 4,605,735 to Miyoshi et al. Methods are also available for synthesizing sulfhydryl-derivatized oligonucleotides which can be reacted with thiol-containing molecules, see, e.g., U.S. Pat. No. 4,757,141 to Fung et al., Connolly et al. (1985) Nucl. Acids Res. 13:4485-4502 and Spoat et al. (1987) Nucl. Acids Res. 15:4837-4848.

A miRNA can include an amine-modified nucleotide, where the nucleotide has been modified to include a reactive amine group. Modified nucleotides can be uridine, adenosine, guanosine, and/or cytosine. For example, the amine-modified nucleotide can be: 5-(3-aminoallyl)-UTP; 8-[(4-amino)butyl]-amino-ATP and 8-[(6-amino)butyl]-amino-ATP; N⁶-(4-amino)butyl-ATP, N⁶-(6-amino)butyl-ATP, N⁴-[2,2-oxy-bis-(ethylamine)]-CTP; N⁶-(6-Amino)hexyl-ATP; 8-[(6-Amino)hexyl]-amino-ATP; or 5-propargylamino-CTP, 5-propargylamino-UTP. Other nucleotides may be similarly modified, for example, 5-(3-aminoallyl)-GTP instead of 5-(3-aminoallyl)-UTP.

A miRNA can be attached to a solid support in a variety of manners. For example, the miRNA may be attached to the solid support by attachment of the 3′ or 5′ terminal nucleotide of the miRNA to the solid support. In some embodiments, the miRNA is attached to the solid support by a linker that serves to distance the miRNA from the solid support. The linker can be at least 15-30 atoms in length, or at least 15-50 atoms in length. The required length of the linker will depend on the particular solid support used. For example, a six atom linker is generally sufficient when high cross-linked polystyrene is used as the solid support.

mRNAs

The plurality of mRNAs that is contacted with the miRNA can be obtained from cells, tissues, organs, or other biological sample that comprises mRNA. Exemplary sources of mRNA are described in more detail below. The plurality of mRNA can be present in a sample, where suitable samples include cell lysates, biological fluids that include mRNAs, tissue homogenates, and the like. The plurality of mRNAs can be isolated, e.g., separated from the source of the mRNAs. In some cases, the mRNAs are purified, e.g., the mRNAs are at least about 50%, at least about 60%, at least about 70%, at least about 75%, at least about 80%, at least about 85%, at least about90%, at least about 95%, at least about 98%, or at least about 99% pure (e.g., free of non-mRNA macromolecules, small molecule contaminants, etc.).

The plurality of mRNAs can include the total mRNA present in a cell, a cell population, a tissue, or other mRNA-containing biological sample.

The plurality of mRNAs can include mRNAs with canonical 5′ seed sequences, and mRNAs lacking canonical 5′ seed sequences. Canonical 5′ seed sequences are nucleotide sequences in an mRNA that are perfectly complementary (100% complementary) by Watson-crick base-pairing to uninterrupted nucleotide sequences 1-7, 2-7, or 2-8 of a miRNA. In some embodiments, an mRNA lacking a canonical 5′ seed sequence contains an imperfect seed which can include an interruption to the canonical seed with either a mismatch or a G:U wobble base-pairing. In some embodiments, an mRNA lacking canonical 5′ seed sequences comprises a nucleotide sequence complementary to nucleotides 4-10, 5-11, 6-12, 7-13, 8-14, 9-15, 10-16, 11-17, 12-18, 13-19 or 9-19 of a miRNA. These alternate complementary sequences in an mRNA may be either 6mers or 7mers encompassing regions outside the originally defined seed region of bases 1-8.

A plurality of mRNAs is contacted with a miRNA under conditions that favor duplex formation (hybridization) between an miRNA and a target mRNA. The miRNA and the target mRNA can have at least about 75%, at least about 80%, at least about 85%, at least about 90%, at least about 95%, at least about 98%, at least about 99%, or 100%, nucleotide sequence identity over a contiguous stretch of from about 10 nucleotides to about 25 nucleotides (nt), e.g., a miRNA and a target mRNA can have at least about 75%, at least about 80%, at least about 85%, at least about 90%, at least about 95%, at least about 98%, at least about 99%, or 100%, nucleotide sequence identity over a contiguous stretch of 10 nt, 11 nt, 12 nt, 13 nt, 14 nt, 15 nt, 16 nt, 17 nt, 18 nt, 19 nt, or 20 nt, or more than 20 nt.

### Hybridization

Conditions that favor duplex formation between an miRNA and a target mRNA are known to those skilled in the art, or can be readily determined by those of ordinary skill in the art. Examples of suitable hybridization solutions include: 1) 80% (v/v) formamide; 0.4 M NaCl; 40 mM piperazine-N,N′-(2-ethanesulfonic acid) (PIPES), pH 6.8; and 1 mM ethylene diamine tetraacetic acid (EDTA); 2) 80% (v/v) formamide; 0.5 M NaCl; 50 mM PIPES, pH 6.4; and 1.25 mM EDTA; and 3) 80% (v/v) formamide; 0.4 M sodium acetate; 40 mM PIPES, pH 6.4; and 1 mM EDTA.

Suitable hybridization temperatures range from about 37 ° C. to about 45° C., e.g., from about 37° C. to about 39° C., from about 39° C. to about 41° C., from about 41° C. to about 43° C., or from about 43° C. to about 45° C.

Hybridization times can range from 1 minute to about 16 hours, e.g., from about 1 minute to about 5 minutes, from about 5 minutes to about 10 minutes, from about 10 minutes to about 15 minutes, from about 15 minutes to about 30 minutes, from about 30 minutes to about 60 minutes, from about 1 hour to about 2 hours, from about 2 hours to about 4 hours, from about 4 hours to about 8 hours, from about 8 hours to about 12 hours, or from about 12 hours to about 16 hours.

The hybridization solution can include one or more additional components, such as a ribonuclease inhibitor. Suitable ribonuclease inhibitors include, e.g., vanadylate ribonucleoside complexes, phenylglyoxal, p-hydroxyphenylglyoxal, polyamines, spermidine, 9-aminoacridine, iodoacetate, bentonite, poly[2′-O-(2,4-dinitrophenyl)]poly(adenyhlic acid), zinc sulfate, bromopyruvic acid, formamide, copper, and zinc. Suitable ribonuclease inhibitors include, e.g., heparin, heparan sulfate, oligo(vinylsulfonic acid), poly(vinylsulfonic acid), oligo(vinylphosphonic acid), and poly(vinylsulfuric acid), or salts thereof. Suitable proteinaceous ribonuclease inhibitors include, e.g., proteinase K, and ribonuclease inhibitor from human placenta. Suitable ribonuclease inhibitors that are chaotropic salts include, e.g., urea salts, guanidine salts, and mixtures thereof. For example, guanidine salts include guanidine thiocyanate or guanidine hydrochloride at a final concentration in the range of about 0.5 M to about 6 M. Suitable ribonuclease inhibitors also include a vanadyl ribonucleoside complex. Other suitable ribonuclease inhibitors are commercially available and include, e.g., RNasin®, RiboLock™, RNAguard™, and the like.

An optional wash step can be included, to remove unbound mRNAs or other materials not bound to the miRNA. The wash solution can be the same as the hybridization solution. Where a miRNA is immobilized on a bead (e.g., a magnetic bead), a magnetic field or a centrifugal force can be applied, to remove the complex comprising the bead, the immobilized miRNA, and any bound mRNA from any unbound materials.

### Elution

After a suitable time, mRNA that has formed a duplex with (e.g., hybridized with) a miRNA is eluted. An mRNA that has formed a duplex with (e.g., hybridized with) a miRNA is also referred to as a “bound mRNA” or a “miRNA-bound mRNA.” Suitable conditions for eluting a bound mRNA from an mRNA:miRNA hybrid include low salt conditions such as Tris (e.g., Tris-HCl) at a concentration of less than about 50 mM (e.g., from about 10 mM Tris-HCl to about 40 mM Tris-HCl) and in a pH range of from about 7 to about 9. For example, a suitable elution solution includes Tris (e.g., Tris-HCl) in a concentration range of from about 50 mM to about 40 mM, from about 40 mM to about 30 mM, from about 30 mM to about 20 mM, or from about 20 mM to about 10 mM, at a pH range of from about 7 to about 9. Bound mRNA can be eluted at a temperature of greater than 42° C., e.g., from about 42° C. to about 95° C., e.g., from about 42° C. to about 45° C., from about 45° C. to about 50° C., from about 50° C. to about 60° C., from about 60° C. to about 70° C., from about 70° C. to about 80° C., from about 80° C. to about 90° C., or from about 90° C. to about 95° C. For example, bound mRNA can be eluted in a low salt buffer that is in the indicated temperature range. The elution solution can include EDTA (e.g., 1 mM EDTA), although in some embodiments, the elution solution does not include EDTA.

In some embodiments, a subject method involves multiple (e.g., two or more) rounds of hybridization and elution. For example, in some embodiments, a subject method involves: a) contacting an miRNA with a plurality of mRNA under conditions that favor duplex formation between the miRNA and at least one member of the plurality of mRNA; b) eluting any bound mRNA (e.g., eluting any mRNA that forms a duplex with the miRNA in step (a)); c) contacting the eluted mRNA from step (b) with the miRNA; and d) eluting any bound mRNA formed in step (c). In some cases, the hybridization conditions in steps (a) and (c) are substantially identical. In some cases, the hybridization conditions in steps (a) and (c) are different from one another, e.g., the hybridization conditions in step (a) are less stringent than the hybridization conditions in step (c), or the hybridization conditions in step (a) are more stringent than the hybridization conditions in step (c). In some cases, the elution conditions in step (b) are different from the elution conditions in step (d). For example, the elution conditions in step (b) include higher salt concentrations and/or higher temperature than the elution conditions in step (d). As another example, the elution conditions in step (b) include lower salt concentrations and/or lower temperature than the elution conditions in step (d).

In some embodiments, a subject method involves multiple (e.g., two or more) rounds of hybridization and elution. For example, in some embodiments, a subject method involves: a) contacting a first miRNA with a plurality of mRNA under conditions that favor duplex formation between the miRNA and at least one member of the plurality of mRNA; b) eluting any bound mRNA (e.g., eluting any mRNA that forms a duplex with the miRNA in step (a)); c) contacting the eluted mRNA from step (b) with a second miRNA that is different from the first miRNA; and d) eluting any bound mRNA formed in step (c). In some cases, the hybridization conditions in steps (a) and (c) are substantially identical. In some cases, the hybridization conditions in steps (a) and (c) are different from one another, e.g., the hybridization conditions in step (a) are less stringent than the hybridization conditions in step (c), or the hybridization conditions in step (a) are more stringent than the hybridization conditions in step (c). In some cases, the elution conditions in step (b) are different from the elution conditions in step (d). For example, the elution conditions in step (b) include higher salt concentrations and/or higher temperature than the elution conditions in step (d). As another example, the elution conditions in step (b) include lower salt concentrations and/or lower temperature than the elution conditions in step (d). In the embodiment described above, step (c) involves contacting the eluted mRNA from step (b) with a second miRNA that is different from the first miRNA, e.g., contacting the eluted mRNA from step (b) with a second miRNA that differs in nucleotide sequence by one or more nucleotides from the first miRNA. In some cases, the first and the second miRNA both have a length of from about 19 nt to about 50 nt. In some cases, the first and the second miRNA differ in length by fewer than 10 nt, e.g., the first and the second miRNA differ in length by 10 nt, 9 nt, 8 nt, 7 nt, 6 nt, 5 nt, 4 nt, 3 nt, 2 nt, or 1 nt. In some cases, the first and the second miRNA have the same length. The first and the second miRNA differ in nucleotide sequence from one another by from 1 nt to about 10 nt, e.g., the first and the second miRNA differ in nucleotide sequence from one another by 1 nt, 2 nt, 3 nt, 4 nt, 5 nt, 6 nt, 7 nt, 8 nt, 9 nt, or 10 nt. In some embodiments, the the first and the second miRNA differ in nucleotide sequence from one another by 1 nt, 2 nt, 3 nt, 4 nt, 5 nt, 6 nt, 7 nt, 8 nt, 9 nt, or 10 nt; and have substantially the same length, or are identical in length. In some embodiments, the first and the second miRNA differ from one another by differential binding to a single nucleotide polymorphism, e.g., the first miRNA binds to a SNP-containing sequence of nucleic acid, and the second miRNA does not bind the SNP-containing sequence.

The eluted mRNA can be stored (e.g., kept at 4° C.; frozen; lyophilized; etc.). The eluted mRNA can also be subjected to any of a number of analytical procedures. For example, the eluted mRNA can be sequenced; the eluted mRNA can be hybridized with a DNA probe; etc. In addition, the eluted mRNA can be used as a template for cDNA synthesis. For example, the mRNA can be used as a template for cDNA synthesis to generate a cDNA; and the cDNA can be further subjected to cloning and/or analytical procedure(s). For example, the cDNA can be sequenced; the cDNA can be cloned into a vector (e.g., a vector that provides for amplification of the copy number of the cDNA; a vector that provides for expression of the cDNA); and the cDNA can be hybridized with a DNA probe.

Sources of mRNAs

Any tissue, cells, organs, or other biological sample that comprises mRNA can be used as a source of mRNA. Suitable sources of mRNA include diseased tissue, cells, and organs. Suitable sources of mRNA include tissue, cells, and organs that are not diseased (e.g., “normal” tissue, cells, and organs).

Cells that may be used as sources of mRNA can be prokaryotic (bacterial cells, including but not limited to those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, Xanthomonas and Streptomyces) or eukaryotic (including fungi (especially yeasts), plants, protozoans, eukaryotic parasites, and animals). Suitable eukaryotic sources of cells that can serve as a source of mRNA include mammalian cells, including rodent cells, lagomorph cells, ungulate cells, human cells, non-human primate cells, etc. A cell that serve as a source of mRNA can be an insect cell, e.g., Drosophila spp. cells, Spodoptera Sf9 and Sf21 cells and Trichoplusa High-Five cells; a nematode cell (e.g., Caenorhabditis elegans cells); or a mammalian cell (e.g., a primary cell), or a mammalian cell line such as COS cells, CHO cells, VERO cells, 293 cells, PERC6 cells, BHK cells, etc.

Suitable tissue sources of mRNA include, but are not limited to, fetal tissues, such as whole fetus or subsections thereof, e.g. fetal brain or subsections thereof, fetal heart, fetal kidney, fetal liver, fetal lung, fetal spleen, fetal thymus, fetal intestine, fetal bone marrow; adult tissues, such as whole brain and subsections thereof, e.g. amygdala, caudate nucleus, corpus callosum, hippocampus, hypothalamus, substantia nigra, subthalamic nucleus, thalamus, cerebellum, cerebral cortex, medula oblongata, occipital pole, frontal lobe, temporal lobe, putamen, adrenal cortex, adrenal medula, nucleus accumbens, pituitary gland, adrenal gland and subsections thereof, such as the adrenal cortex and adrenal medulla, aorta, appendix, bladder, bone marrow, colon, colon proximal with out mucosa, heart, kidney, liver, lung, lymph node, mammary gland, ovary, pancreas, peripheral leukocytes, placental, prostate, retina, salivary gland, small intestine, skeletal muscle, skin, spinal cord, spleen, stomach, testis, thymus, thyroid gland, trachea, uterus, and uterus without endometrium. The tissue can be a human tissue, or a non-human mammalian tissue.

The tissues can be from normal and disease or condition states of the same organism or multiple organisms, where disease or condition states include, e.g., cancer;

stroke; heart failure; aging; infectious diseases; inflammation; exposure to toxic, drug or other agents; conditional treatment, such as heat shock, sleep deprivation, physical activity, etc.; different developmental stages; and the like.

Mammalian somatic cells are suitable sources of mRNAs. Mammalian somatic cells that are suitable sources of mRNA include blood cells (reticulocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells). Mammalian germ cells (spermatocytes and oocytes) can also be used as sources of mRNA, as can the progenitors, precursors and stem cells that give rise to the above somatic and germ cells. Also suitable for use as mRNA sources are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

Non-limiting examples of suitable cells from which mRNA can be obtained are cells of multicellular organisms, e.g., cells of invertebrates and vertebrates, such as myoblasts, neutrophils, erythrocytes, osteoblasts, chondrocytes, basophils, eosinophils, adipocytes, invertebrate neurons, vertebrate neurons, mammalian neurons, adrenomedullary cells, melanocytes, epithelial cells, and endothelial cells; tumor cells of all types (e.g., melanoma, myeloid leukemia, carcinomas of the lung, breast, ovaries, colon, kidney, prostate, pancreas and testes); cardiomyocytes, endothelial cells, lymphocytes (T-cell and B cell), mast cells, vascular intimal cells, hepatocytes, leukocytes including mononuclear leukocytes; stem cells such as hematopoietic stem cells, neural, skin, lung, kidney, liver and myocyte stem cells; osteoclasts, connective tissue cells, keratinocytes, melanocytes, hepatocytes, and kidney cells. Suitable cells also include known cell lines, including, but not limited to, Jurkat T cells, NIH3T3 cells, CHO, COS, etc. Cell lines include those found in ATCC Cell Lines and Hybridomas (8^{th} ed, 1994, or latest edition, or on the world wide web at www(dot)atcc(dot)org), Bacteria and Bacteriophages (19^{th} ed., 1996), Yeast (1995), Mycology and Botany (19^{th} ed., 1996), and Protists: Algae and Protozoa (18^{th} ed., 1993), available from American Type Culture Co. (Manassas, Va.).

Suitable mammalian cells include primary cells and immortalized cell lines. Primary cells include primary cells used in limited passaging. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), C2C12 cells (ATCC No. CRL-1772), human embryonic kidney (HEK) cells (ATCC No. CRL1573), HLHepG2 cells, and the like.

In some embodiments, the cell is a neuronal cell or a neuronal-like cell. The cells can be of human, non-human primate, mouse, or rat origin, or derived from a mammal other than a human, non-human primate, rat, or mouse. Suitable cell lines include, but are not limited to, a human glioma cell line, e.g., SVGp12 (ATCC CRL-8621), CCF-STTG1 (ATCC CRL-1718), SW 1088 (ATCC HTB-12), SW 1783 (ATCC HTB-13), LLN-18 (ATCC CRL-2610), LNZTA3WT4 (ATCC CRL-11543), LNZTA3WT11 (ATCC CRL-11544), U-138 MG (ATCC HTB-16), U-87 MG (ATCC HTB-14), H4 (ATCC HTB-148), and LN-229 (ATCC CRL-2611); a human medulloblastoma-derived cell line, e.g., D342 Med (ATCC HTB-187), Daoy (ATCC HTB-186), D283 Med (ATCC HTB-185); a human tumor-derived neuronal-like cell, e.g., PFSK-1 (ATCC CRL-2060), SK-N-DZ (ATCCCRL-2149), SK-N-AS (ATCC CRL-2137), SK-N-FI (ATCC CRL-2142), IMR-32 (ATCC CCL-127), etc.; a mouse neuronal cell line, e.g., BC3H1 (ATCC CRL-1443), EOC1 (ATCC CRL-2467), C8-D30 (ATCC CRL-2534), C8-S (ATCC CRL-2535), Neuro-2a (ATCC CCL-131), NB41A3 (ATCC CCL-147), SW10 (ATCC CRL-2766), NG108-15 (ATCC HB-12317); a rat neuronal cell line, e.g., PC-12 (ATCC CRL-1721), CTX TNA2 (ATCC CRL-2006), C6 (ATCC CCL-107), F98 (ATCC CRL-2397), RG2 (ATCC CRL-2433), B35 (ATCC CRL-2754), R3 (ATCC CRL-2764), SCP (ATCC CRL-1700), OA1 (ATCC CRL-6538).

Suitable mRNA includes mRNA obtained from cells that are exposed to an external or internal signal. External and internal signals (stimuli) include, but are not limited to, infection of a cell by a microorganism, including, but not limited to, a bacterium (e.g., Mycobacterium spp., Shigella, Chlamydia, and the like), a protozoan (e.g., Trypanosoma spp., Plasmodium spp., Toxoplasma spp., and the like), a fungus, a yeast (e.g., Candida spp.), or a virus (including viruses that infect mammalian cells, such as human immunodeficiency virus, foot and mouth disease virus, Epstein-Barr virus, and the like; viruses that infect plant cells; etc.); change in pH of the medium in which a cell is maintained or a change in internal pH; excessive heat relative to the normal range for the cell or the multicellular organism; excessive cold relative to the normal range for the cell or the multicellular organism; an effector molecule such as a hormone, a cytokine, a chemokine, a neurotransmitter; an ingested or applied drug; a ligand for a cell-surface receptor; a ligand for a receptor that exists internally in a cell, e.g., a nuclear receptor;

hypoxia; a change in cytoskeleton structure; light; dark; a mitogen, including, but not limited to, lipopolysaccharide (LPS), pokeweed mitogen; stress; antigens; sleep pattern (e.g., sleep deprivation, alteration in sleep pattern, and the like); an apoptosis-inducing signal; electrical charge (e.g., a voltage signal); ion concentration of the medium in which a cell is maintained, or an internal ion concentration, exemplary ions including sodium ions, potassium ions, chloride ions, calcium ions, and the like; presence or absence of a nutrient; metal ions; a transcription factor; a tumor suppressor; cell-cell contact; adhesion to a surface; peptide aptamers; RNA aptamers; intrabodies; genetic modification; and the like.

Isolation of mRNA

Isolation of mRNA can be readily performed using techniques well known to those of skill in the art. For example, chromatographic methods can be used to separate or isolate nucleic acids from protein or other cell components such as lipids, polysaccharides, and the like. Suitable methods can involve electrophoresis with a gel matrix, filter columns, alcohol precipitation, and/or chromatographic methods. For example, mRNA can be isolated from cells using methods generally involve lysing the cells with a chaotropic agent (e.g., guanidinium isothiocyanate) and/or a detergent (e.g., N-lauroyl sarcosine). A population of mRNA can be purified, e.g., by gel electrophoresis, column chromatography, or other well-known method.

Sub-Populations of mRNA

A plurality of mRNA for use in a subject method can include a non-selected population of mRNA, or a selected population (a “sub-population” or “subset”) of mRNA. A population of mRNA for use in a subject method can be pre-selected, e.g., a total mRNA population can be subjected to one or more processing steps to isolate a sub-population of mRNA (e.g., a “subset” of mRNA).

As one non-limiting example, an initial population of mRNA isolated from a cell(s), tissue, organ, or other biological sample (diseased or normal) can be selected to include or exclude poly(A)⁺ mRNA. Selection of poly(A)⁺ mRNA can be achieved by contacting an initial population of mRNA (comprising both poly(A)⁺ and poly(A)⁻ mRNA) with immobilized oligo(dT). Where a sub-population of poly(A)⁺ mRNA is desired, poly(A)⁺ mRNA bound to the immobilized oligo(dT) is eluted, resulting in a sub-population of poly(A)⁺ mRNA (e.g., e.g., at least about 50%, at least about 60%, at least about 70%, at least about 80%, at least about 90%, or more than 90% of the sub-population of mRNA is poly(A)⁺). Where a sub-population of poly(A)⁻ mRNA is desired, the sub-population of mRNA that does not bind to the immobilized oligo(dT) is collected; this sub-population comprises poly(A)⁻ mRNA (e.g., at least about 50%, at least about 60%, at least about 70%, at least about 80%, at least about 90%, or more than 90% of the sub-population of mRNA is poly(A)⁻).

In addition, the cells used as a source of mRNA can be pre-sorted on the basis of expression of a cell surface marker, expression of a detectable label (e.g., expression of a fluorescent protein such as a green fluorescent protein), to generate a sub-population of cells (e.g., a selected population; a sorted population). The cells can be sorted using fluorescence activated cell sorting, use of magnetic beads (e.g., magnetic beads comprising an antibody specific for a cell surface marker), and the like. The sub-population of cells can be used as a source of mRNA.

As another non-limiting example, an initial population of mRNA can be subjected to subtractive hybridization, to exclude one or more species of mRNA. Subtractive hybridization methods are known in the art. For example, a first population of mRNA isolated from a first tissue or cell(s), where the first tissue or cell(s) is a diseased tissue or cell(s), can be subjected to subtractive hybridization using a second population of mRNA (or a cDNA copy thereof) isolated from a second tissue or cell(s), where the second tissue or cell(s) is of the same tissue type or cell type as the first tissue or cell(s), and where the second tissue or cell(s) is not diseased, e.g., does not have the same disease as the first tissue or cell(s).

As another non-limiting example, an initial population of mRNA can be subjected to selection based on hybridization to a nucleic acid array. For example, an initial population of mRNA can be hybridized to an array of nucleic acid probes; and a sub-population of mRNA that does not hybridize to the array can be used in a subject method (e.g., can be contacted with a miRNA). Alternatively, an initial population of mRNA can be hybridized to an array of nucleic acid probes; the sub-population that hybridizes to the array can be eluted from the array; and the eluted sub-population of mRNA can be used in a subject method (e.g., can be contacted with a miRNA).

As another non-limiting example, an initial population of mRNA can be subjected to selection based on hybridization to a miRNA. For example, an initial population of mRNA can be hybridized to a first immobilized miRNA; and a sub-population of mRNA that does not bind to the first miRNA can be used in a subject method (e.g., can be contacted with a second miRNA, where the second miRNA differs in nucleotide sequence from the first miRNA by 1 nt to 5 nt, by 5 nt to 10 nt, by 10 nt to 20 nt, or more than 20 nt). Alternatively, an initial population of mRNA can be hybridized to a first immobilized miRNA; and any bound mRNA can be eluted, where the eluted mRNA is used in a subject method (e.g., is contacted with a second miRNA, where the second miRNA differs in nucleotide sequence from the first miRNA by 1 nt to 5 nt, by 5 nt to 10 nt, by 10 nt to 20 nt, or more than 20 nt).

As another non-limiting example, an initial population of mRNA can be subjected to size selection. For example, an initial population of mRNA comprising mRNA species of various lengths (e.g., from about 30 nt to about 5000 kb) can be size-selected to generate one or more sub-populations in which at least about 50%, at least about 60%, at least about 70%, at least about 80%, at least about 90%, or more than 90% of the mRNA in a given sub-population has a length within a selected length range. Exemplary length ranges that can be included in a sub-population include, e.g., from about 30 nt to about 100 nt, from about 100 nt to about 500 nt, from about 500 nt to about 1000 nt (1 kilobase (kb)), from about 1 kb to about 5 kb, from about 1 kb to about 10 kb, from about 5 kb to about 10 kb, from about 10 kb to about 50 kb, from about 50 kb to about 100 kb, from about 100 kb to about 1000 kb, from about 1000 kb to about 2000 kb, from about 2000 kb to about 3000 kb, from about 3000 kb to about 4000 kb, or from about 4000 kb to about 5000 kb.

### Detectable Labels

The mRNA can be detectably labeled. For example, mRNA can be detectably labeled before contacting with an miRNA. Alternatively, in some cases, only the eluted mRNA is detectably labeled.

By “detectably labeled” is meant that the mRNA comprises a member of a signal producing system and is thus detectable, either directly or through combined action with one or more additional members of a signal producing system.

Examples of directly detectable labels include isotopic and fluorescent moieties incorporated into, usually covalently bonded to, a moiety of the mRNA, such as a nucleotide monomeric unit, or a photoactive or chemically active derivative of a detectable label which can be bound to a functional moiety of the mRNA. Isotopic moieties or labels of interest include ³²P, ³³P, and the like. Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy3 and Cy5, macrocyclic chelates of lanthanide ions, e.g. quantum dye™, fluorescent energy transfer dyes, such as thiazole orange-ethidium heterodimer, TOTAB, etc. Also of interest are nanometer sized particle labels detectable by light scattering, e.g. “quantum dots.”

Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. Additional labels of interest include those that provide for signal only when the mRNA with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

There are several methods and compositions known for derivatizing nucleic acids with reactive functionalities which permit the addition of a label. For example, several approaches are available for biotinylating nucleic acids so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al. Nucl. Acids Res. (1985) 13:1529-1541 which discloses biotinylation of the 5′ termini of a nucleic acid via an aminoalkylphosphoramide linker arm. Several methods are also available for synthesizing amino-derivatized nucleic acids which are readily labeled by fluorescent or other types of compounds derivatized by amino-reactive groups, such as isothiocyanate, N-hydroxysuccinimide, or the like, see, e.g., Connolly (1987) Nucl. Acids Res. 15:3131-3139, Gibson et al. (1987) Nucl. Acids Res. 15:6455-6467 and U.S. Pat. No. 4,605,735 to Miyoshi et al. Methods are also available for synthesizing sulflhydryl-derivatized nucleic acids which can be reacted with thiol-specific labels, see, e.g., U.S. Pat. No. 4,757,141 to Fung et al., Connolly et al. (1985) Nuc. Acids Res. 13:4485-4502 and Spoat et al. (1987) Nucl. Acids Res. 15:4837-4848. A comprehensive review of methodologies for labeling nucleic acids is provided in Matthews et al., Anal. Biochem. (1988) 169:1-25.

For example, a nucleic acid may be fluorescently labeled by linking a fluorescent molecule to the non-ligating terminus of the nucleic acid. Guidance for selecting appropriate fluorescent labels can be found in Smith et al., Meth. Enzymol. (1987) 155:260-301; Karger et al., Nucl. Acids Res. (1991) 19:4955-4962; Haugland (1989) Handbook of Fluorescent Probes and Research Chemicals (Molecular Probes, Inc., Eugene, Oreg.). Exemplary fluorescent labels include fluorescein and derivatives thereof, such as disclosed in U.S. Pat. No. 4,318,846 and Lee et al., Cytometry (1989) 10:151-164, and 6-FAM, JOE, TAMRA, ROX, HEX-1, HEX-2, ZOE, TET-1 or NAN-2, and the like.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-Dctp.

All of the mRNA of the plurality of mRNA can comprise the same detectable label. Alternatively, two or more members of the plurality of mRNAs can comprise two or more different detectable labels, which are distinguishable one from the other.

Examples of distinguishable labels are well known in the art and include: two or more different emission wavelength fluorescent dyes, such as Cy3 and Cy5, two or more isotopes with different energy of emission, e.g., ³³P and 32P, gold or silver particles with different scattering spectra, labels which generate signals under different treatment conditions, like temperature, pH, treatment by additional chemical agents, etc., or generate signals at different time points after treatment.

Processing and Analysis of Eluted mRNA

The eluted mRNA can be subjected to any of a number of analytical procedures. The eluted mRNA is considered a candidate target mRNA for the miRNA. Validation of a candidate target can be carried out using any of a variety of assays, including, e.g., a luciferase assay; a protein blot assay; a target protector assay; and an assay in a transgenic mouse model.

As one example, the eluted mRNA can be sequenced; the eluted mRNA can be hybridized with a DNA probe; etc. In addition, the eluted mRNA can be used as a template for cDNA synthesis. For example, the mRNA can be used as a template for cDNA synthesis to generate a cDNA; and the cDNA can be further subjected to cloning and/or analytical procedure(s). For example, the cDNA can be sequenced; the cDNA can be cloned into a vector (e.g., a vector that provides for amplification of the copy number of the cDNA; a vector that provides for expression of the cDNA); and the cDNA can be hybridized with a DNA probe (e.g., the cDNA can be contacted with a probe array).

In some embodiments, a subject method involves: a) contacting an miRNA with a plurality of mRNA under conditions that favor hybridization between the miRNA and at least one member of the plurality of mRNA; b) eluting any bound mRNA, e.g., eluting any mRNA that forms a duplex with the miRNA; and c) sequencing the eluted mRNA.

In some embodiments, a subject method involves: a) contacting an miRNA with a plurality of mRNA under conditions that favor hybridization between the miRNA and at least one member of the plurality of mRNA; b) eluting any bound mRNA, e.g., eluting any mRNA that forms a duplex with the miRNA; and c) synthesizing a cDNA copy of the eluted mRNA, using the eluted mRNA as a template for cDNA synthesis. In some embodiments, a subject method involves: a) contacting an miRNA with a plurality of mRNA under conditions that favor hybridization between the miRNA and at least one member of the plurality of mRNA; b) eluting any bound mRNA, e.g., eluting any mRNA that forms a duplex with the miRNA; c) synthesizing a cDNA copy of the eluted mRNA, using the eluted mRNA as a template for cDNA synthesis; and d) sequencing the cDNA. In some embodiments, a subject method involves: a) contacting an miRNA with a plurality of mRNA under conditions that favor hybridization between the miRNA and at least one member of the plurality of mRNA; b) eluting any bound mRNA, e.g., eluting any mRNA that forms a duplex with the miRNA; c) synthesizing a cDNA copy of the eluted mRNA, using the eluted mRNA as a template for cDNA synthesis; and d) cloning the cDNA in a vector, where suitable vectors include expression vectors.

In some embodiments, a subject method involves: a) contacting an miRNA with a plurality of mRNA under conditions that favor hybridization between the miRNA and at least one member of the plurality of mRNA; b) eluting any bound mRNA, e.g., eluting any mRNA that forms a duplex with the miRNA; c) synthesizing a cDNA copy of the eluted mRNA, using the eluted mRNA as a template for cDNA synthesis; and d) contacting the cDNA with a probe array. In these embodiments, the cDNA is detectably labeled.

In some embodiments, the eluted mRNA population is used as template for synthesizing cDNA copies of the eluted mRNA; and the cDNA is detectably labeled. Detectable labels that are suitable for use for labeling a cDNA are described above.

In some embodiments, a subject method involves hybridizing a plurality of eluted mRNAs with an array of nucleic acid probes (a “probe array” or “nucleic acid array” or “nucleic acid probe array”).

Probe array are ordered macroarrays or microarrays of nucleic acid molecules (probes) that are fully complementary to, partially complementary to, or identical to, an eluted mRNA (or a cDNA copy of an eluted mRNA), and that are positioned on a support material in a spatially separated organization. Macroarrays can be sheets of nitrocellulose or nylon upon which probes have been spotted. Microarrays position the nucleic acid probes more densely such that up to 10,000 nucleic acid molecules can be fit into a small region, e.g., a region of from about 1 cm² to about 4 cm². Microarrays can be fabricated by spotting nucleic acid molecules, e.g., DNA, onto substrates or fabricating oligonucleotide sequences in situ on a substrate. Spotted or fabricated nucleic acid molecules can be applied in a high density matrix pattern of up to about 30 non-identical nucleic acid molecules per s cm or higher, e.g. up to about 100 or even 1000 per cm². Microarrays can be fabricated using coated glass as the solid support. By having an ordered array of mRNA-binding nucleic acid molecules (probes), the position of each sample can be tracked and linked to the original sample. A variety of different array devices in which a plurality of distinct nucleic acid probes are stably associated with the surface of a solid support are known to those of skill in the art. Suitable substrates for arrays include nylon, glass and silicon. Such arrays may vary in a number of different ways, including average probe length, sequence or types of probes, nature of bond between the probe and the array surface, e.g. covalent or non-covalent, and the like.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

The probe array can include from about 10 to about 10⁹ different probes, e.g., from about 10 to about 10⁹ nucleic acid probes, each of which has a different nucleotide sequence from the other probes in the array. For example, a probe array can include from about 10 to 10², from about 10² to about 10³, from about 10³ to about 10⁴, from about 10⁴ to about 10⁵, from about 10⁵ to about 10⁶, from about 10⁶ to about 10⁷, from about 10⁷ to about 10⁸, or from about 10⁸ to about 10⁹ different probes. “Different probes” refers to probes differing in nucleotide sequence from one another.

Any given probe in an array can have a length of from about 10 nucleotides (nt) to about 100 nt, e.g., each probe in an array can independently have a length of 10 nt, 11 nt, 12 nt, 13 nt, 14 nt, 15 nt, 16 nt, 17 nt, 18 nt, 19 nt, 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the probes are DNA probes having a length of from about 20 nt to about 25 nt.

A probe can be “addressable,” e.g., the nucleotide sequence, or perhaps other physical or chemical characteristics, of a probe can be determined from its address, i.e. a one-to-one correspondence between the sequence or other property of the probe and a spatial location on, or characteristic of, the solid phase support to which it is attached. For example, an address of a probe can be a spatial location, e.g. the planar coordinates of a particular region containing copies of the probe.

In some cases, each of the probe spots in an array comprising a nucleic acid probe correspond to the same kind of gene; i.e. genes that all share some common characteristic or can be grouped together based on some common feature, such as species of origin, tissue or cell of origin, functional role, disease association, etc. For example, each of the different probe nucleic acids in the different probe spots on the array are of the same type, i.e. that are coding sequences of the same type of gene. As such, the arrays of this embodiment will be of a specific array type. A variety of specific array types are provided by the subject invention. Specific array types of interest include: human, cancer, apoptosis, cardiovascular, cell cycle, hematology, mouse, human stress, mouse stress, oncogene and tumor suppressor, cell-cell interaction, cytokine and cytokine receptor, disease-related arrays, signaling cascade arrays, tissue-specific arrays, cell type-specific arrays, rat, rat stress, blood, mouse stress, neurobiology, and the like. An array can also include nucleic acid probes comprising single nucleotide polymorphisms (SNP). For example, an array can include a first probe comprising a first nucleotide sequence and a second probe comprising a second nucleotide sequence, where the first and second nucleotide sequences differ only in that the first or the second nucleotide sequence includes a SNP. Arrays designed to determine copy number variation and/or alterations in splicing can also be used. As noted above, the “address” information can include information regarding the specific type of probe included in a particular spot. Suitable arrays also include a single nucleotide polymorphism array, a splice variant array, a copy number variation array, a regulatory nucleic acid array, and the like.

A probe array includes a solid phase support (“substrate”), which may be planar or a collection of microparticles, that carries or carry probes as described above fixed or immobilized, e.g., covalently, at specific addressable locations. For example, a subject array includes a solid phase support having a planar surface, which carries a plurality of nucleic acids, each member of the plurality comprising identical copies of an oligonucleotide or polynucleotide probe immobilized to a fixed region, which does not overlap with those of other members of the plurality. Typically, the nucleic acid probes are single stranded and are covalently attached to the solid phase support at known, determinable, or addressable, locations. The density of non-overlapping regions containing nucleic acids in a microarray is typically greater than 100 per cm², e.g., greater than 1000 per cm². An array may have the form of a biochip, a multiwell device, and the like. An array can have a probe density of greater than about 60, 100, 600, 1000, 5,000, 10,000, 40,000, 100,000, or 400,000 different oligonucleotide probes per cm².

The substrates (solid phase support) of the arrays may be fabricated from a variety of materials. The materials from which the substrate is fabricated should ideally exhibit a low level of non-specific binding during hybridization events. In some cases, it the material will be transparent to visible and/or UV light. The solid phase support can be flexible or rigid. For flexible substrates, materials of interest include: nylon, both modified and unmodified, nitrocellulose, polypropylene, and the like. For rigid substrates, suitable materials include: glass (e.g., silicon dioxide); plastics, e.g. polytetrafluoroethylene, polypropylene, polystyrene, polycarbonate, and blends thereof, and the like; metals, e.g. gold, platinum, and the like; etc. Also of interest are composite materials, such as glass or plastic coated with a membrane, e.g. nylon or nitrocellulose, etc.

Hybridization between a probe and a test nucleic acid (where a test nucleic acid includes an eluted mRNA, or a cDNA copy of an eluted mRNA, or an amplicon generated using an eluted mRNA as a template, or an amplicon generated using a cDNA copy of an eluted mRNA as a template) results in a “readout,” where “readout” refers to a parameter, or parameters, which are measured and/or detected that can be converted to a number or value. In some contexts, readout may refer to an actual numerical representation of such collected or recorded data. For example, a readout of fluorescent intensity signals from an array is the address and fluorescence intensity of a signal being generated at each hybridization site of the array; thus, such a readout may be registered or stored in various ways, for example, as an image of the array, as a table of numbers, or the like. The “readout” can provide the identity of the bound probe to which a test nucleic acid binds.

The total number of spots on the substrate will vary depending on the number of different oligonucleotide probe spots (oligonucleotide probe compositions) one wishes to display on the surface, as well as the number of non probe spots, e.g., control spots, orientation spots, calibrating spots and the like, as may be desired. The pattern present on the surface of the array can include at least 2 distinct nucleic acid probe spots, at least about 5 distinct nucleic acid probe spots, at least about 10 distinct nucleic acid spots, at least about 20 nucleic acid spots, or at least about 50 nucleic acid spots.

In some cases, it may be desirable to have each distinct probe spot or probe composition be presented in duplicate, i.e. so that there are two duplicate probe spots displayed on the array for a given target. In some cases, each target represented on the array surface is only represented by a single type of oligonucleotide probe. In other words, all of the oligonucleotide probes on the array for a give target represented thereon have the same sequence. In certain embodiments, the number of spots will range from about 200 to 1200. The number of probe spots present in the array can make up a substantial proportion of the total number of nucleic acid spots on the array, where in many embodiments the number of probe spots is at least about 25 number %, at least 50 number %, at least about 80 number %, or at least about 90 number % of the total number of nucleic acid spots on the array.

An array can be prepared using any convenient means. One means of preparing an array is to first synthesize the oligonucleotides for each spot and then deposit the oligonucleotides as a spot on the support surface. The oligonucleotides may be prepared using any convenient methodology, where chemical synthesis procedures using phosphoramidite or analogous protocols in which individual bases are added sequentially without the use of a polymerase, e.g. such as is found in automated solid phase synthesis protocols, where such techniques are well known to those of skill in the art.

Test nucleic acids include an eluted mRNA(s), or a cDNA copy of an eluted mRNA(s), or an amplicon generated using an eluted mRNA as a template, or an amplicon generated using a cDNA copy of an eluted mRNA as a template. An eluted mRNA is generated as described above. A cDNA copy, or an amplicon, can be generated by methods known in the art. Eluted mRNA can be labeled and used directly as a test nucleic acid, or converted to a labeled cDNA test nucleic acid. mRNA can be labeled non-specifically (randomly) directly using chemically, photochemically or enzymatically activated labeling compounds. Methods for generating labeled cDNA probes are known in the art, and include the use of oligonucleotide primers and labeled nucleotide triphosphate(s). Primers that may be employed include oligo dT, random primers, e.g. random hexamers and gene specific primers.

Test nucleic acids are contacted with the probe array under nucleic acid hybridization conditions, where such conditions can be adjusted, as desired, to provide for an optimum level of specificity in view of the particular assay being performed. Suitable hybridization conditions are well known to those of skill in the art and reviewed in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; Sambrook, J. and Russell, W., Molecular Cloning: A Laboratory Manual, Third Edition, Cold Spring Harbor Laboratory Press, Cold Spring Harbor (2001); and WO 95/21944. In some cases, stringent hybridization conditions are used, i.e. conditions that are optimal in terms of rate, yield and stability for specific probe-test nucleic acid hybridization and provide for a minimum of non-specific probe/test nucleic acid interaction. Stringent conditions are known to those of skill in the art.

Those skilled in the art can readily analyze data generated using an array. Methods of analyzing data generated using an array include those described in, e.g., WO 9743450; WO 03023058; WO 03022421; WO 03029485; WO 03067217; WO 03066906; WO 03076928; WO 03093810; and WO 03100448. For example, binding of an eluted mRNA (or a cDNA copy thereof) is readily detected using a method that detects a label associated with the mRNA or cDNA.

For example, hybridization is performed by first exposing the array with a prehybridization solution. Next, the array is incubated under binding conditions with a solution containing mRNAs (or cDNA copies or amplicons thereof) for a suitable binding period. Binding conditions will vary depending on the application and are selected in accordance with the general binding methods known including those referred to in: Maniatis et al., Molecular Cloning: A Laboratory Manual (1989), 2nd Ed., Cold Spring Harbor, N.Y. and Berger and Kimmel, Methods in Enzymology, Volume 152, Guide to Molecular Cloning Techniques (1987), Academic Press, Inc., San Diego, Calif.; Young and Davis (1983) Proc. Natl. Acad. Sci. (U.S.A.) 80: 1194, which are incorporated herein by reference. In some embodiments, the solution may contain about 1 molar of salt and about 1 to 50 nanomolar of targets (e.g., mRNA or cDNA). Finally, the array is washed with a buffer, e.g., the hybridization buffer, to remove the unbound targets. In some embodiments, the cavity is filled with the buffer after washing the sample. Thereafter, the array can be aligned on a detection or imaging system. The detection or imaging system is capable of qualitatively analyzing the reaction between the probes and targets (e.g., bound mRNA or cDNA). Based on this analysis, sequence information of the targets (e.g., bound mRNA or cDNA) is extracted.

### Differential Analysis

The methods described can be used to detect differences (e.g., sequence differences) between two samples. Specifically contemplated applications include identifying and/or quantifying differences between mRNA from a sample that is normal and from a sample that is not normal or between two differently treated samples, as described above. In addition, mRNA can be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. Such a sample can be compared to a cell that is normal with respect to that disease or condition. Phenotypic traits include symptoms of, or susceptibility to, a disease or condition of which a component is or may or may not be genetic.

As one non-limiting example, a single nucleotide polymorphism (SNP) associated with a disease or disorder can be detected. As an example, a SNP that affects miRNA binding can be detected. As another example, differences in copy number or alterations in splicing or transcription levels can alter binding of target mRNA to a particular miRNA; as such, differences in copy number, alterations in splicing, and alterations in transcription levels can be detected.

### Validation Assays

Validation of a candidate target can be carried out using any of a variety of assays, including, e.g., a luciferase assay; a protein blot assay; a target protector assay; overexpression of an miRNA (wild-type or mutant sequence) in an isolated cell in vitro or in an animal model system; knockdown of an miRNA in an isolated cell in vitro or in an animal model system; Argonaute precipitation; and an assay in a transgenic mouse model.

For example, a transgenic mouse model comprising a transgene that comprises a nucleotide sequence encoding a particular miRNA can be used to analyze the effect of the miRNA on the level of a candidate target mRNA. As another example, a construct comprising a nucleotide sequence encoding a candidate mRNA-luciferase mRNA hybrid can be used to assess the effect of a particular miRNA on a candidate mRNA, where any effect of the miRNA on the level of the candidate mRNA can be assessed using an assay to detect luciferase activity. Alternatively, the effect of an miRNA on a candidate mRNA can be assessed by detecting the level of a protein encoded by the candidate mRNA. Detection of the level of a protein encoded by a candidate mRNA can be carried out using any of a variety of well-known assays, including protein blots (using an antibody specific for the protein encoded by the candidate mRNA), enzyme-linked immunosorbent assays, enzyme assays (e.g., where the protein encoded by the candidate mRNA is an enzyme), and the like. The effect of an miRNA on a candidate mRNA can be assessed by use of target protector nucleic acids.

### Utility

A subject method is useful for identifying an mRNA target of a miRNA. Identification of an mRNA target of an miRNA is useful in a variety of research and diagnostic applications, including, e.g.: in analysis of development of an organism; in analysis of the effect of a single nucleotide polymorphism; in analysis of mRNAs expressed in diseased tissue; in analysis of regulation of gene expression (e.g., regulation of translation) by an miRNA; etc. For example, once the target(s) of a given miRNA are identified, the miRNA can be used to design therapeutic nucleic acids that modulate translation of the target mRNA(s), e.g., to ameliorate a disease condition. As one non-limiting example, where a given miRNA is determined to target an mRNA that regulates angiogenesis, the miRNA can be used to design therapeutic nucleic acids that modulate angiogenesis (e.g., to decrease angiogenesis in the context of tumor growth; or to increase angiogenesis in the context of wound healing). As another example, once the target(s) of a given miRNA are identified, target protector nucleic acids can be designed that hybridize to the region on a target mRNA that is bound by the miRNA, thereby modulating regulation of the target mRNA by the miRNA. Such target protector nucleic acids can be used in research and therapeutic applications.

As one non-limiting example, where a given miRNA is determined to target a functional class of targets via a non-canonical seed, a mutant miRNA specifically affecting these subset of targets can be used to therapeutically target mRNAs affected by base-pairing to the non-canonical seed.

### Kits

The present invention provides kits for carrying out a subject method. A subject kit can comprise an array, where the array can comprise a pattern of probes on a planar support or be incorporated into a multiwell configuration, biochip configuration, or other configuration. A subject kit can further comprise one or more additional reagents for use in the assay to be performed with the array, where such reagents include: reagents for isolating mRNAs; reagents for detectably labeling a nucleic acid; reagents used in the binding step, e.g. hybridization buffers; signal producing system members, e.g. substrates; control probes, e.g., pre-labeled control probes; washing and/or hybridization containers; and the like. In some embodiments, a subject kit comprises one or more reagents for one or more of: a) modifying a nucleic acid; b) labeling a nucleic acid with a detectable label; and c) attaching a nucleic acid to a solid support.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Experimental Procedures

### Biochemical Screen

3′ end amine-modified miR-1 was biotinylated according to manufacturer's instructions (Pierce) and incubated overnight at room temperature with 10 μg of RNA from 6-8-week-old mouse hearts under ribonuclease protection assay hybridization conditions (Current Protocols). Pull-downs were performed with streptavidin Dynabeads M-280. Associated mRNAs were eluted with low salt and heating according to manufacturer's instructions and used for cDNA synthesis or labeled for hybridization to Affymetrix mouse 430 version 2.0 expression arrays. Samples were spiked with exogenous controls (Applied Biosystems) for normalizing input and eluate signal intensities. Three different experimental hybridizations were used for array analyses and experiments have been performed numerous times for reproducibility.

### Cell Culture, Transfections, and Luciferase Assays

Luciferase assays were performed with 90% confluent HeLa S3 cells in 24-well plates. The cells were transfected with 0.4 μg of luciferase construct with 0.04 μg of Renilla and 10 or 50 pmols of miRNA using Lipofectamine 2000. Luciferase activity was measured 24 hours after transfection and normalized to Renilla activity. For knockdown and competitor studies, confluent HL-1 cells were transfected with 2′-O-methyl antisense miR-1 or target protectors by Amaxa nucleofection and harvested 48 hours later for gene expression analysis.

### Gene Expression Analysis

mRNA and miRNA levels were detected with the ABI 7900HT real-time PCR system (Applied Biosystems). Standard western blotting methods were used on RIPA heart lysates of 6-8-week-old mice. Cpeb1, Rgs19, Smyd3, Mib1, and Ncx1 antibodies were from Abcam. Camk2d rabbit polyclonal antibody was from Novus. Kcnd2, cdk6, KRas, and Ocrl antibodies were from Santa Cruz Biotechnology. Kcnq1 antibody was from Sigma.

### Transgenic Mice

The ∝-MHC-miR-1-2 mice used in this study were generated by inserting pre-miR-1 with approx 300 base pairs of flanking sequence and has been described before (Zhao et al., 2005).

### Electrocardiography

Acquisition and analysis of electrocardiograms were performed as described previously (Zhao et. al., 2007). Briefly, mice were anesthetized with 1.75% isoflurane in 2 L/min O2 at a core body temperature of 37-38° C. 6-lead ECGs were recorded at 10KHz using a Dual Bio Amp signal conditioner and a PowerLab 4/30 ADC (AD Instruments). Data analysis was performed offline using the software package Chart5Pro (v 5.4.2, AD Instruments). Each interval was measured with electronic calipers on 50-150 signal-averaged beats from each lead, and then averaged to get a single set of intervals for each mouse. The QRS interval was measured from the onset of the Q-wave to the isoelectric point preceding the first, rapid repolarization wave. The QT interval was measured from the onset of the Q-wave to the end of the second, slower repolarization wave. The measured QT interval was corrected for heart rate using the rodent correction formula QTc=QT/(RR/100)^{0.5} obtained by (Mitchell et al., 1998). Mean, standard deviation, and standard error of the mean were calculated for each genotype using these intervals. All statistical comparisons were made with T-tests on two independent samples assuming a two-tailed distribution for each parameter.

### Bioinformatics and Statistical Analysis

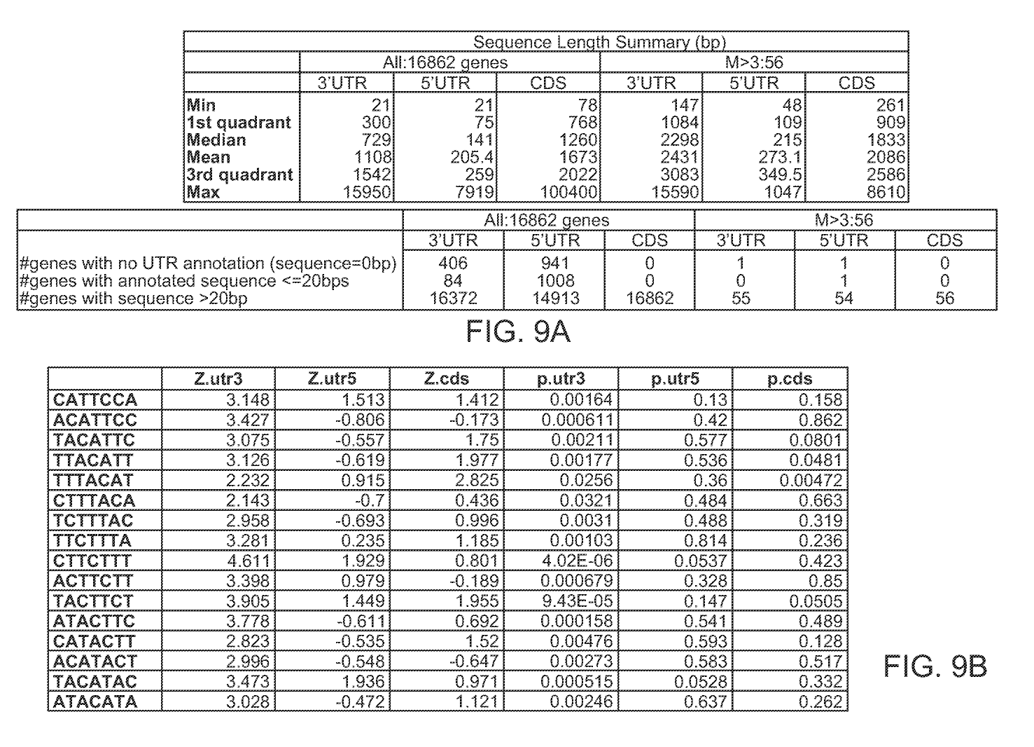

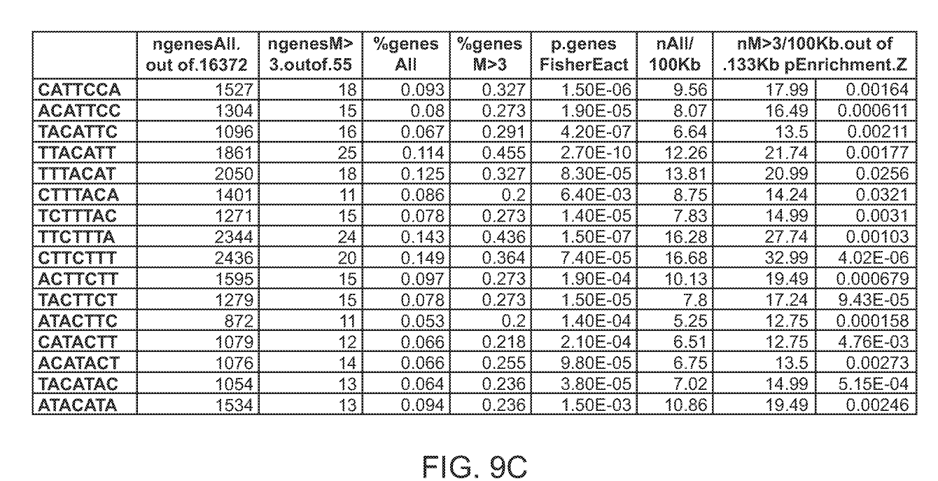

Affymetrix probeset IDs were mapped to EntrezeGene ID using Affymetrix's annotation (version na24.mm8). For each unique EntrezGene, the most extreme M value (log2 ratio) among corresponding probesets and the longest 3′ UTR among corresponding RefSeq transcripts were used for heptamer analysis and are summarized in FIG. 9A. In-house scripts were used to extract 5′ UTR, CDS, and 3′ UTR sequences from NCBI RefSeq database (release 26), as well as for the analysis of their heptamer content. For each heptamer, we assessed its enrichment in the M>3 genes compared to the background heptamer density in all 3′ UTRs using a Z-score, defined as Z=(N_{M>3}−N_{bg})/sqrt(N_{bg}), where N_{M>3} is the heptamer count in the 3′ UTRS of M>3 genes, and N_{bg} is the expected heptamer count under the background frequency. Accompanying two-sided p-values were derived from Gaussian distribution. In addition, empirical p-values, p*, were derived from the empirical distribution of Z-scores of 4898 miRNA-associated heptamers.

### RESULTS

Biochemical Screen for miRNA Targets

Our biochemical screen was based on sequence complementarity between miRNA and potential mRNA targets without bias to the region of sequence matching. To identify miR-1-interacting cardiac mRNAs, we biotinylated a synthetic miR-1 containing an amine at the 3′ end; 3′ end labeling was done to avoid steric hindrance of the 5′ end of the miRNA, which usually participates in complementary binding with a cognate target mRNA. mRNA isolated from adult mouse heart tissue was used as input for hybridization with biotinylated miR-1 followed by streptavidin-mediated pull-down. Binding stringency was optimized in small-scale by monitoring the pull-down and elution of putative targets by cDNA synthesis and RT-PCR of the eluate. In this screen, mRNA transcripts were isolated from mouse heart and were thus present in the same relative abundance as occurs in vivo, theoretically mimicking endogenous competition for hybridization to the miRNA.

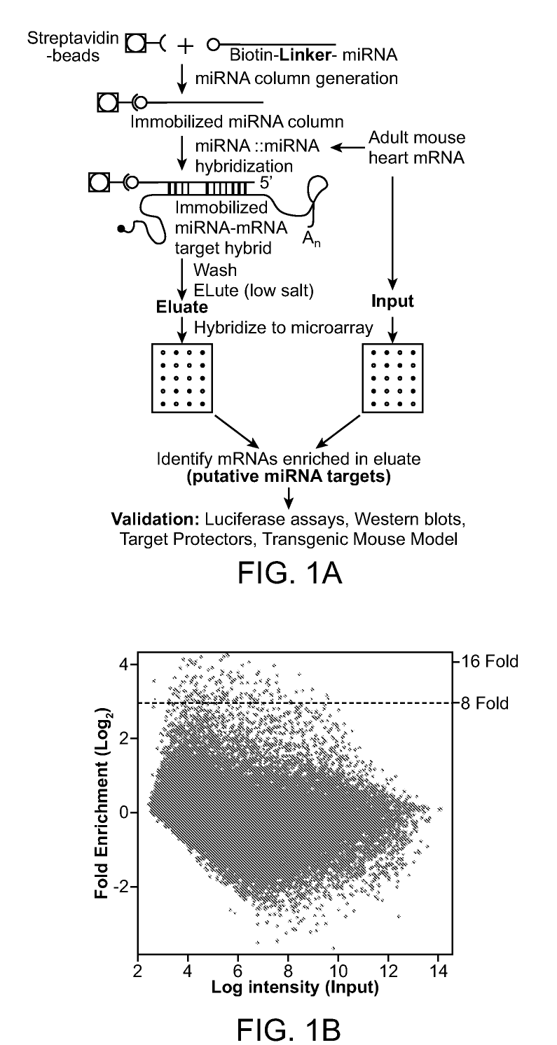

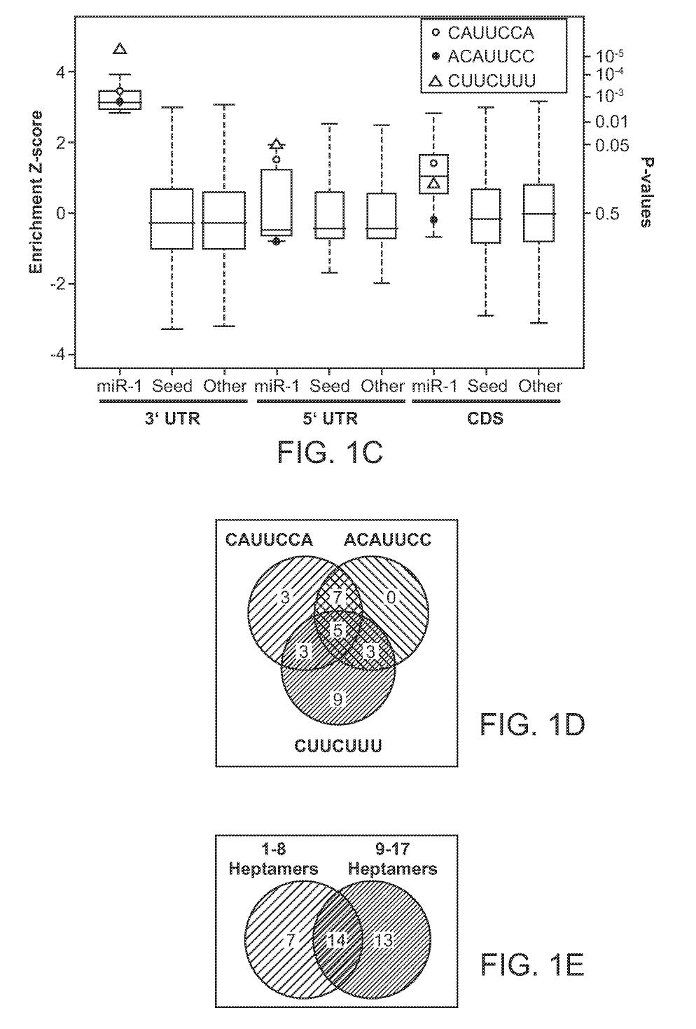

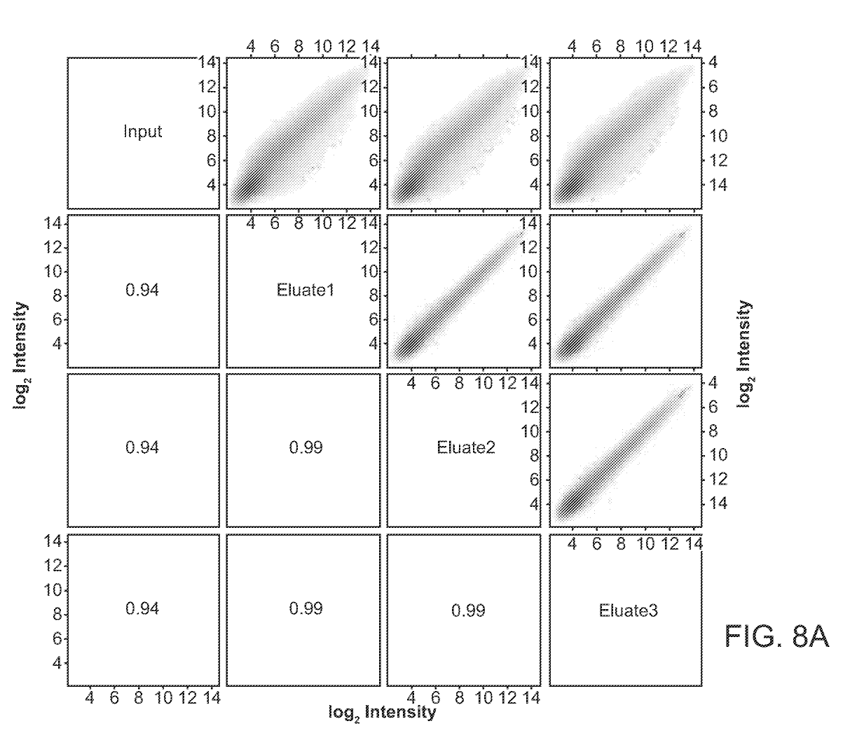

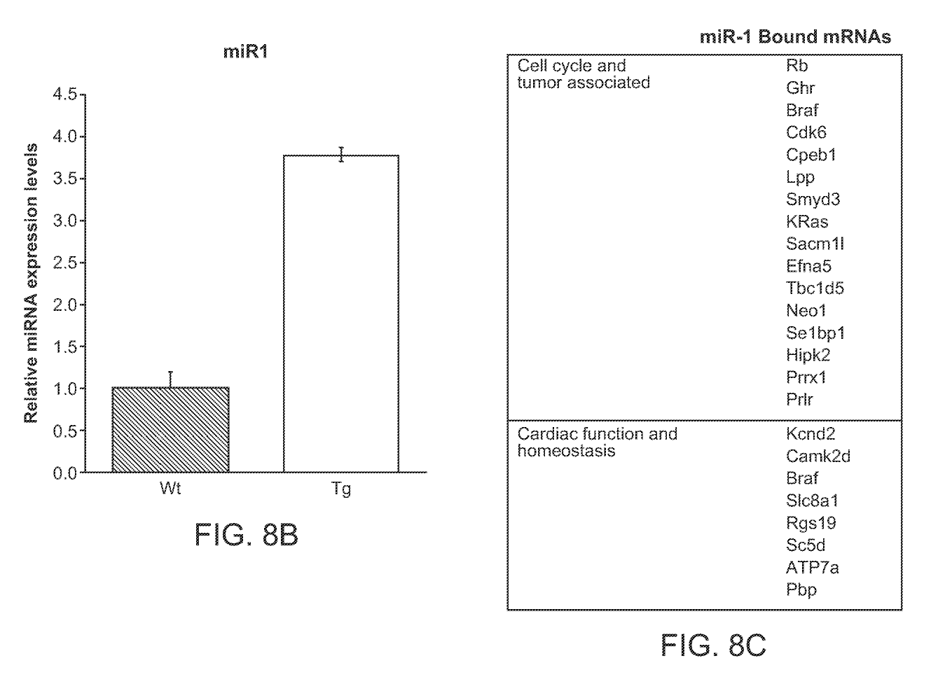

Unbound mRNAs were removed by serial washes, and miR-1-bound mRNAs were eluted from streptavidin-conjugated magnetic beads with a low-salt buffer (FIG. 1A). To reduce the background noise in the hybridization assay, equal amounts of eluate and input mRNA were labeled and hybridized to Affymetrix expression (mRNA) arrays to evaluate the enrichment of eluted transcripts compared to input (FIG. 1A). Eluates from three independent experiments were used for arrays and had a high degree of correlation with one another (FIG. 8A), allowing robust statistical analysis of the data. The relative intensities of an mRNA in the eluate vs. input allowed us to calculate the fold enrichment of a transcript (Y-axis, FIG. 1B). This was compared to the relative input intensity, a measure of relative abundance (X-axis, FIG. 1B). Interestingly, relatively rare transcripts competed successfully with highly abundant transcripts for hybridization to the column, as indicated by the number of low-abundance mRNAs enriched ≧8-fold (FIG. 1B). At the opposite end of the spectrum, a discrete set of highly abundant cardiac transcripts was also enriched in the eluate, indicating a broad range of binding sensitivity in this assay.

FIG. 1. Biochemical Screen for miR-1 Targets. (A) Schematic of biochemical screen to identify miR-1 targets in adult mouse heart. Biotinylated synthetic miR-1 was hybridized with mRNAs isolated from adult mouse hearts (Input). Pulldown eluate of miR-1-associated mRNAs was hybridized to an Affymetrix chip and compared to Input mRNA intensities on an Affymetrix chip in triplicate. (B) A plot of Input (X-axis) vs log₂ M (M=fold enrichment) revealed 55 unique targets enriched ≧8-fold and enrichment of rare transcripts. Only a discrete set of abundant mRNAs was highly enriched; most enriched targets were moderately expressed. The enrichment (M) in Y-axis allows determination of targets that were bound above background in the column. (C) Significant enrichment of all the miR-1 specific heptamers in the 3′ UTRs of putative targets was observed in the miR-1 pulldown eluate. The Y-axis indicates the two-sided p-value from the normal distribution. “miR-1” summarizes the 16 heptamers complementary to the miR-1 sequence. The miR-1 specific 5′ heptamers CAUUCCA (red circle, nts 1-7) and ACAUUCC (green circle, nts 2-8), complementary to the first eight bases of miR-1, were significantly enriched (miR-1) compared to the summary of occurrence of any of the other 683 heptamers complementary to the first eight bases of any of the 416 mouse miRNAs found in miRBase v10.0 (“Seed”). “Other” summarizes 4201 heptamers complementary to non-seed regions of any miRNA other than miR-1. Boxes contain the middle half of the data (1^{st} quartile, median, 3^{rd} quartile) and the hinges are 1.5 IQR (inter-quartile range=height of the box). The heptamer CUUCUUU (blue triangle) complementary to bases 9-15 of miR-1 showed the strongest enrichment (p*=0.0012 compared to all miRNA-associated heptamers from the empirical distribution shown above). (D) Distributions of the classic 5′ seeds and the sequence with the highest enrichment in putative targets with enrichment ≧8-fold. CAUUCCA is complementary to bases 1-7 of miR-1; ACAUUCC: bases 2-7; CUUCUUU: bases 9-15. Among the 12 genes that have both CAUUCCA and ACAUUCC, eight have the full 8-mer ACAUUCCA. (E) Distribution of the classic 5′ seeds (complementary to bases 1-7 or 2-8 of miR-1) and the enriched region (complementary to bases 9-15, 10-16, or 11-17) of miR-1 in putative targets with ≧8-fold enrichment.

FIG. 8. Additional validation of pull-down and in vivo mouse model. (A) High reproducibility of the pulldown assay. The scattermatrix plot of log₂ intensities from the four arrays shows that the three eluate arrays are concordant to each other and different from the input array. The numbers inside the boxes below each diagonal indicate the correlation coefficient (r) for the pairwise comparisons. (B) Quantification of representative myocardial expression of miR-1 in α-MHC-miR-1-2 transgenic hearts by qRT-PCR. (C) Table representative of functions of putative targets of miR-1 in the adult mouse myocardium.

Statistical Analysis of miRNA Target Screen

To assess the sequence specificity of the putative mRNA targets isolated from our hybridization screen, we performed a systematic sequence enrichment analysis for all possible 4,898 heptamers complementary to any of the 461 known murine miRNAs documented in miRBase v.10.0. This included 683 heptamers complementary to the 5′ seed regions (defined as nts 1-7 or 2-8) of any miRNA and 4,215 heptamers corresponding only to non-5′seed regions of any miRNAs. For each heptamer, a normalized z-score and associated p-value were calculated to evaluate the sequence enrichment in the 3′ UTRs of all 16,372 unique genes represented on the affymetrix array (FIGS. 9A-C). The same analysis was also performed for the 5′ UTRs and coding sequences.

To analyze a manageable set of putative mRNA targets isolated from the hybridization screen, we used a relatively stringent criterion of ≧8-fold enrichment, which yielded 55 unique annotated transcripts. Consistent with the hybridization-based approach, all of the 16 miR-1 associated heptamers were significantly enriched in the 3′ UTRs of the top 55 putative targets (p-values for the enrichment z-score for the miR-1 associated heptamers ranged from 4×10⁻⁶ to 0.03, FIG. 1C and FIG. 9B). Each of the 16 miR-1 associated heptamers was more likely to be found in the 3′ UTRs of the top 55 putative targets than were other heptamers from the full set of 4898 miRNA-associated heptamers (empirical p=0.0012 to 0.05, FIG. 9C). Among the miR-1 heptamers, the classic 5′ seed sequences corresponding to nts 1-7 and 2-8 were highly enriched (p=0.0016 and 0.0006; empirical p=0.015 and 0.008) (FIGS. 9B and 9C), suggesting that the screen was effective in enriching for mRNA transcripts with 5′ seed matches. Surprisingly, several other heptamers also occurred frequently and were significantly enriched, among which “cuucuuu”, corresponding to bases 9-15 of miR-1, was the most enriched (p=4.02×10⁻⁶; empirical p(p*)=0.012) (FIG. 1C). Occurrence of matches to nts 2-8 and 9-17 were highest in 3′ UTRs but were not significantly enriched in 5′ UTRs or coding sequences (FIGS. 1C and 9B).

The 3′ UTRs frequently contained miR-1 complementary sequences for both 5′ and middle regions (FIG. 1D). Of the 55 enriched transcripts, 21 (38%) had one or more heptamer seed matches complementary to the 5′ end of miR-1 (nt 1-7, 2-8, or 1-8) (FIG. 1D). By comparison, 13.6% of random mRNAs would be expected to have a miR-1 seed match (Fisher's exact test p-value<10⁻⁵). Of the 21 transcripts, 14 also had sequence matching to the mid-region of miR-1 defined above. Interestingly, 13 of 55 transcripts had heptamer matches to nts 9-17 of miR-1, but no 5′ seeds (nt 1-7, 2-8, or 1-8) (FIG. 1E). This initial evaluation of the biochemical screen for miRNA targets suggested that we were, at a minimum, enriching for transcripts with sequence complementarity to miR-1.

FIG. 9. Summary statistics for miR-1 pull-down. (A) Table of data set used for preprocessing. The 45101 probesets on the Mouse Genome 430 2.0 array were mapped to 16,862 unique EntrezGene Ids with Refseq annotation using the annotation from Affymetrix (version na24.mm8, Nov. 5, 2007). Each of the 16,862 genes is associated with an M value, average log₂ ratio of the miR-1 elude relative to the control, and one 3′UTR, 5′UTR, CDS sequence from corresponding Refseq transcripts (Release 26, Nov. 20, 2007) from NCBI GenBank. The maximum M (for 8316 genes with multiple probesets) or the longest sequence (for 2242 genes with multiple Refseq transcripts) was chosen as representative feature for analysis. Hence, as a result, the 3′UTR, 5′UTR, and CDS sequences associated with a gene might not necessarily be derived from the same transcript. Sequences 20 bp or shorter were also excluded, as were NM_{—}207659 (no UTR annotation) and NM_{—}03092 (5′UTR=8 bp) for M>3. (B) Summary statistics for all 16 miR-1 hepatmers in 3′UTRs, 5′UTRs, and coding regions when normalized to 683 5′ seed and 4215 non 5′ seed miRNA associated heptamers. (z: enrichment z-score; p:

two-sided p-value of the z-score). (C) Summary statistics for all 16 miR-1 heptamers in 3′UTRs of M>3 genes using all 4̂7=16384 heptamers as the “null.”

Experimental Validation of Putative miRNA Targets

Several experimentally documented miR-1 targets were enriched in this screen but did not meet the high threshold (≧8-fold) we set for validation studies. These included connexin 43 (C×43) and the potassium channel Kir2.1, both of which are implicated in miR-1's regulation of the cardiac conduction system (Yang et al., 2007). Hand2 (Zhao et al., 2005) was not isolated in this screen as it is predominantly expressed in the early embryo and is present at very low levels in the adult heart.

We also tested the validity of several transcripts with 5′ seed matches isolated from the screen (≧8-fold enrichment) that have not been demonstrated to be regulated by miR-1. To validate putative miR-1 targets in vivo, we generated transgenic mice expressing miR-1 in the postnatal heart using the α-myosin heavy chain promoter (α-MHC-miR-1). Transgenic mice with high levels of expression developed severe dilated cardiomyopathy within 1 month but lower expressors (˜3-5 fold vs. wildtype) survived to reproductive age, allowing creation of stable lines (FIG. 8B). Conversely, we used 2′-O-methyl antisense oligonucleotides to achieve knockdown of miR-1 function in the mouse HL-1 atrial cardiomyocyte cell line, HL-1.

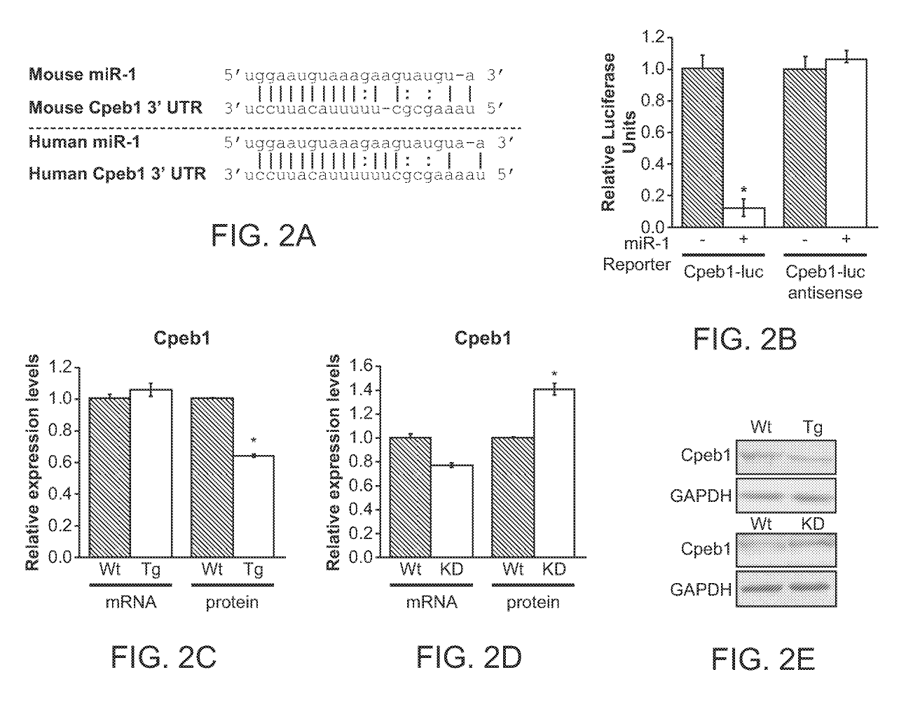

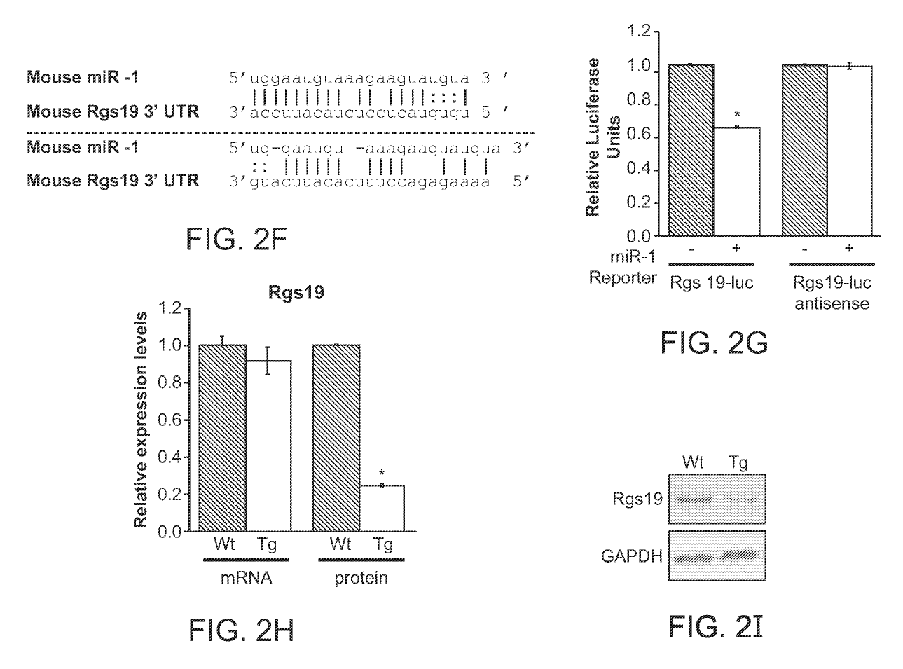

The first set of targets we tested were those predicted by bioinformatic approaches. The transcript in our screen with the highest enrichment (>12-fold) that overlapped with Targetscan was Cpeb1, a translational regulator of cell-cycle-regulated genes (reviewed in Richter, 2007). A conserved miR-1 binding site with an extended 5′ seed (nt 2-13) (FIG. 2A) conferred miR-1-responsive repression of luciferase activity (FIG. 2B). Cpeb1 protein, but not mRNA levels, was modestly decreased in α-MHC-miR-1 hearts (FIGS. 2C and 2E), where Cpeb1 was likely already subject to repression by miR-1. However, knockdown of miR-1 in HL-1 cells increased Cpeb1 protein levels (FIGS. 2D and 2E). Thus, Cpeb1 is likely a direct target of miR-1, consistent with miR-1's putative cell-cycle regulatory function (Zhao et al., 2007; Zhao et al., 2005).

Another putative mRNA target enriched in this screen was Rgs19, a regulator of G-protein signaling (Berman et al., 1996). It had two 5′ seed matches (one perfect and another with G:U wobbles) in the mouse but was not conserved in humans and therefore was not predicted by some algorithms (FIG. 2F). Insertion of the 3′ UTR of Rgs19 into the luciferase 3′ UTR significantly reduced luciferase activity in a miR-1 dependent manner (FIG. 2G). Rgs19 protein levels were not detectable in western blots of HL-1 cell lysates. However, in α-MHC-miR-1 hearts, Rgs19 protein was markedly decreased without any change in mRNA levels, consistent with miR-1-dependent translational repression (FIGS. 2H and 2I).

FIG. 2. Experimental Validation of miR-1 Target Screen. (A) miR-1 complementary sequence in mouse and human Cpeb1 mRNA. (B) Relative luciferase activity of a constitutively active reporter with tandem copies of the predicted Cpeb1 miR-1 binding sequence inserted in sense orientation into the luciferase 3′ UTR shows miR-1 mediated repression. (C, D) Quantification of Cpeb1 protein levels shows downregulation in transgenic mice with 3-fold excess miR-1 (Tg) without a concomitant decrease in mRNA levels (qRT-PCR). Knockdown (KD) of miR-1 with 2′-O-methyl-antisense oligo led to elevated levels of Cpeb1 protein. (E) Representative Western blots quantified in (C, D). (F) Sequence complementarity of two neighboring regions in the mouse Rgs19 3′ UTR with miR-1. The first site has a perfect 5′ seed with classic Watson-Crick base-pairing indicated with bars. The second 5′ seed is imperfect with two complementary G:U wobbles indicated colons. (G) Relative luciferase activity repression conferred by Rgs19 miR-1 binding sites in sense but not antisense orientation. (H) Quantification of Rgs19 protein levels in miR-l-expressing transgenic mice (Tg) without changes in mRNA levels by qRT-PCR. (I) Representative Western blot of Rgs19 in Tg hearts. Error bars represent standard deviation and asterisks indicate p<0.05.

Validation of miR-1 Targets with Imperfect 5′ Seed Matching

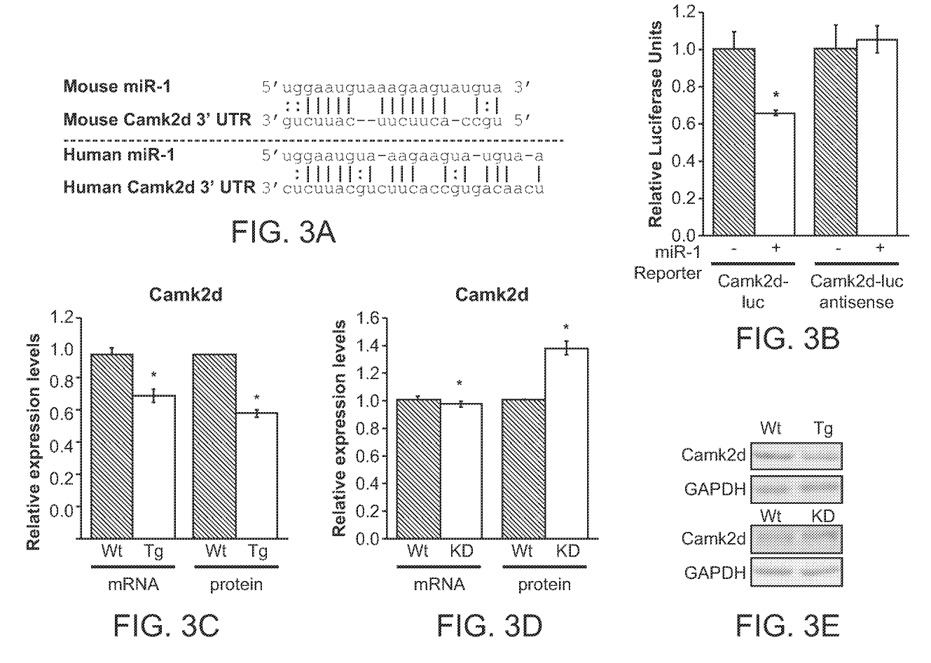

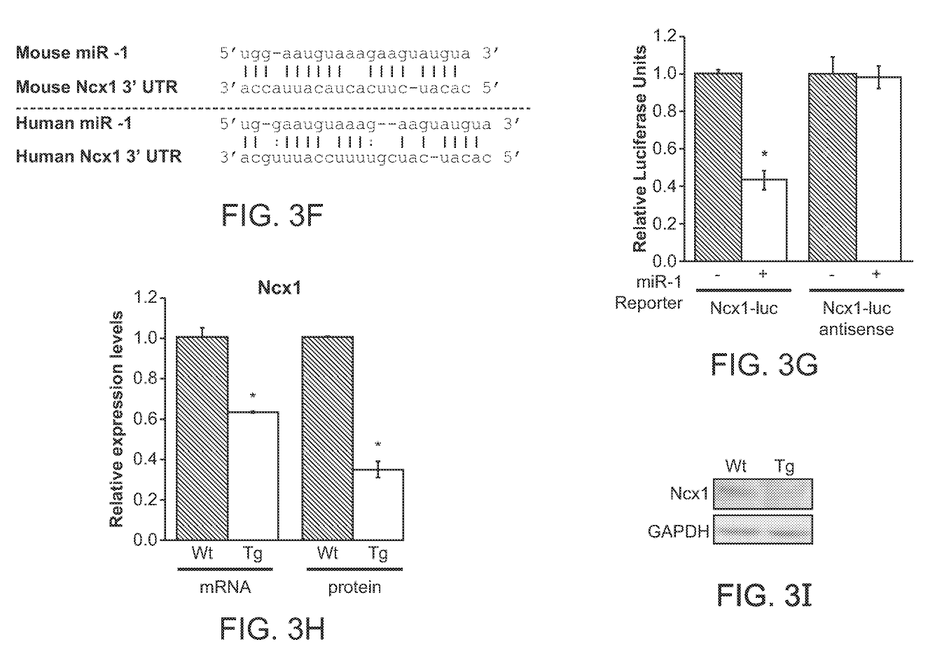

Because <40% of the most enriched mRNAs from our screen contained a canonical 5′ seed match with miR-1, we searched for interrupted 5′ seed matches with compensatory base-complementarity to miR-1 outside the 5′ region. Imperfect 5′ matches with various degrees of compensatory base pairing throughout the rest of the miRNA-binding site were found in ˜80% of targets. One such target, calcium/calmodulin protein kinase II delta (Camk2d), was enriched >13-fold in our biochemical screen but had not been predicted to be regulated by miR-1. Camk2d plays a central role in synchronizing excitation-contraction coupling by phosphorylating several proteins involved in calcium-induced calcium release (reviewed in Bers, 2002) and also represses cardiac hypertrophy (Backs et al., 2006). The putative miR-1 binding site was in the 3′ UTR near the stop codon and contained a partial 5′ seed match but had a perfect sequence match with nts 10-16 of miR-1 (FIG. 3A). This binding site conferred repression in a heterologous luciferase reporter assay (FIG. 3B). Camk2d protein and mRNA levels were both downregulated in α-MHC-miR-1 mice (FIGS. 3C and 3E). Reciprocally, Camk2d protein levels were increased upon miR-1 knockdown, suggesting that this site is a true miR-1 target (FIGS. 3D and 3E).

Another highly enriched target that lacked an intact 5′ seed was the Na/Ca pump, Ncx1. Ncx1 is the primary Na/Ca exchanger in the heart responsible for calcium export at the end of each contraction cycle, allowing muscle fibers to relax during diastole (reviewed in Bers, 2002). A putative miR-1 binding site with a mismatch to the 5′ seed but with significant complementarity with the rest of miR-1 (FIG. 3F) conferred repression in luciferase assays (FIG. 3G). α-MHC-miR-1 hearts revealed a sharp reduction in Ncx1 protein (FIGS. 3H and 3I).

FIG. 3. Validation of Novel miR-1 Targets with Non-canonical 5′ Seeds. (A) Potential miR-1 binding site in mouse and human Camk2d 3′ UTR possessing partial 5′ base-pairing with miR-1 but a complementary heptameric seed in mouse corresponding to bases 10-16 of miR-1. (B) Repression of luciferase activity by miR-1 upon insertion of binding site in (A) into luciferase 3′ UTR (Camk2d-luc) in sense orientation. (C, D) Quantification of Camk2d mRNA and protein levels in (C) α-MHC miR-1-expressing transgenic hearts (Tg) relative to wild-type (Wt) littermates or (D) in HL-1 cells with knockdown (KD) of miR-1. (E) Representative Western blots of Camk2d protein in Tg hearts or KD HL-1 cells compared to Wt. GAPDH represents loading control. (F) Complementarity of imperfect miR-1 binding site in mouse and human Ncx1 3′ UTR. (G) Luciferase activity in the presence or absence of miR-1 when binding site cloned downstream of luciferase as tandem repeats (Ncx1-luc) in the sense or antisense orientation. (H) Quantification of mRNA and protein levels in Tg hearts relative to Wt. (I) Representative Western blot of Ncx1 protein in Wt or Tg hearts. Error bars represent standard deviation and asterisks indicate p<0.05.

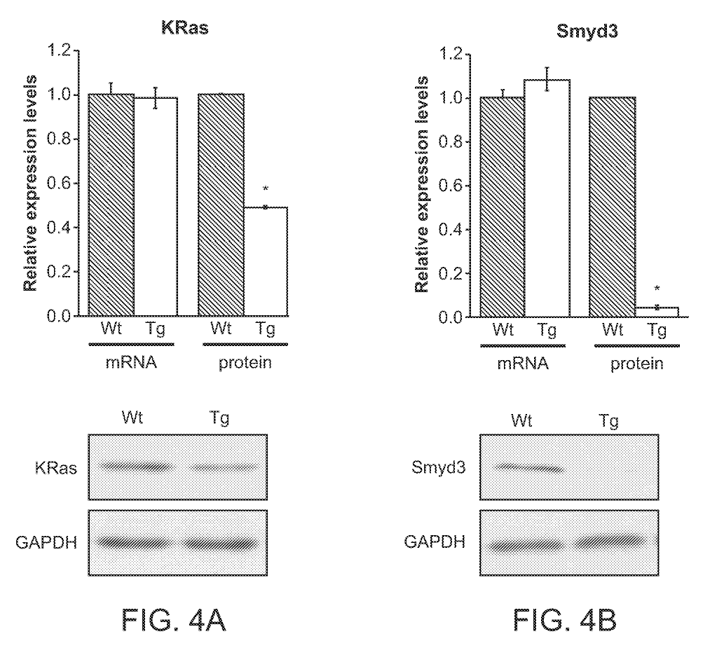

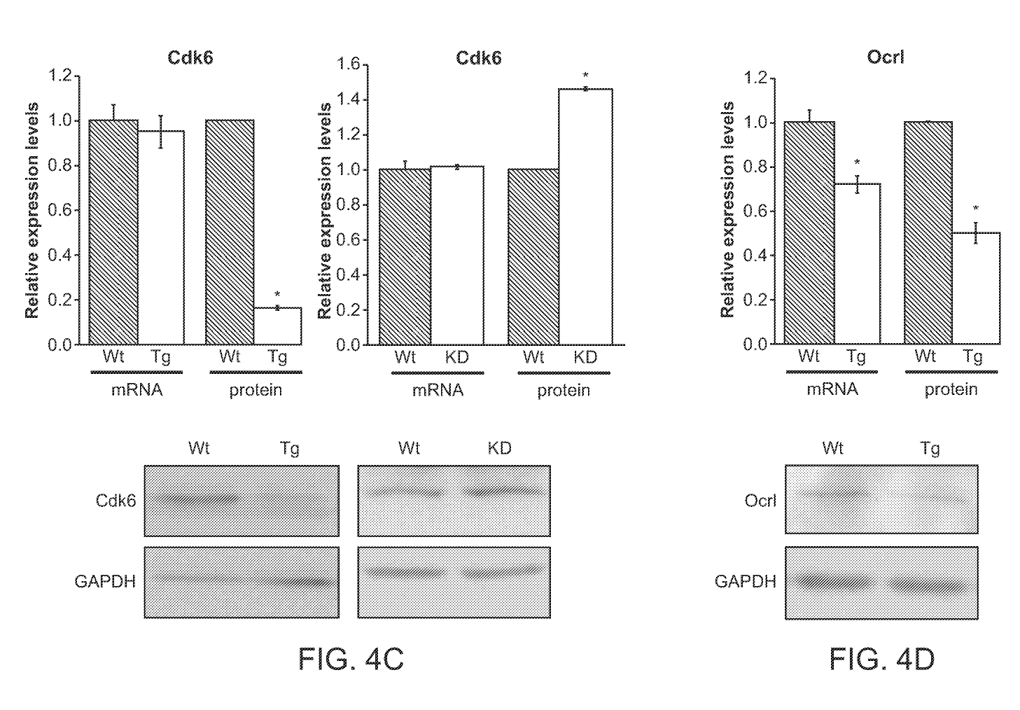

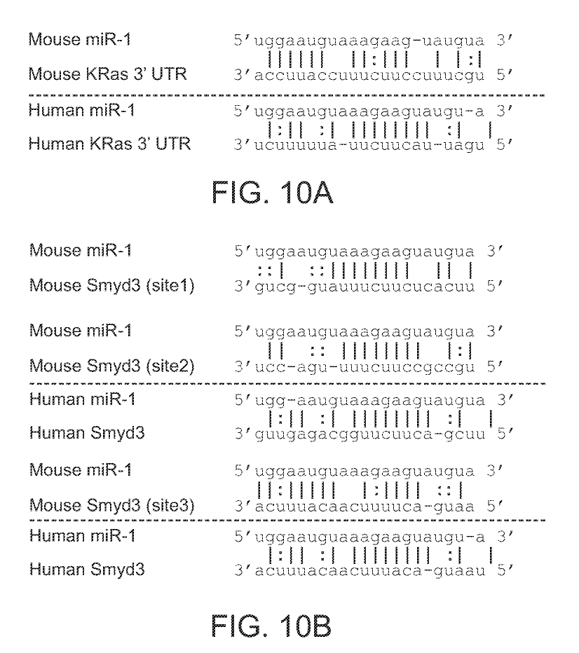

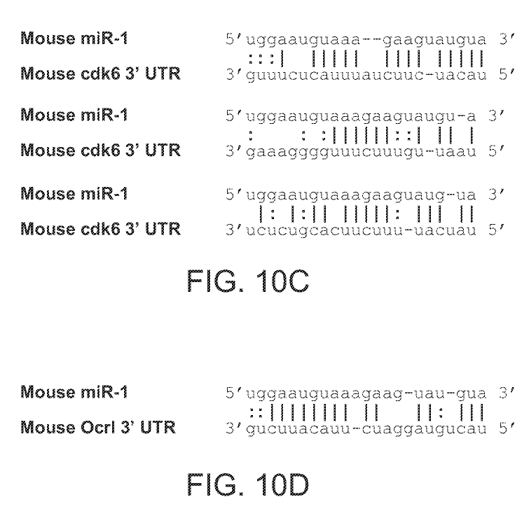

Many of the transcripts with an imperfect 5′ match that were enriched >8-fold in our screen are involved in cell-cycle control, a physiological function of miR-1 (Zhao et al., 2007; Zhao et al., 2005). For example, K-Ras, which contributes to cancer pathogenesis (Dinulescu et al., 2005; Haigis et al., 2008; Johnson et al., 2001; Kumar et al., 2007) and has been implicated in Noonan syndrome, characterized by congenital cardiac defects and growth defects (Pandit et al., 2007) was enriched 8.3 fold in our pull-down eluate. K-Ras has hexameric 5′ base-pairing (nts 2-7 of miR-1) and compensatory base complementarity to nts 10-15 of miR-1 (FIG. 10A). K-Ras protein levels were decreased in α-MHC-miR-1 mice (FIG. 4A) and modestly increased by knockdown of miR-1 in HL-1 cells. Smyd3, a methyltransferase whose activity has oncogenic potential (Hamamoto et al., 2004), was almost undetectable by Western blot in miR-1 transgenic hearts, but mRNA level were equivalent to wild type hearts (FIG. 4B). Similarly, Cdk6, a cyclin-dependent kinase whose protein levels must be downregulated to exit the cell cycle and enable differentiation (reviewed in Malumbres and Barbacid, 2005), was severely reduced at the protein level in miR-1 transgenic mice but not at the mRNA level (FIG. 4C). Cdk6 protein levels were modestly upregulated upon knockdown of miR-1 in HL-1 cells (FIG. 4C). Finally, another transcript with extensive but imperfect 5′ base-pairing (FIG. 10D) that was enriched in our screen was oculocerebrorenal syndrome of Lowe protein (Ocrl). Protein levels were downregulated in miR-1 transgenic mice, consistent with it being an in vivo miR-1 target (FIG. 4D). Thus, our approach was effective in identifying several novel miR-1 targets with perfect or imperfect 5′ seed matches.

FIG. 4. Validation of Enriched miR-1 Targets Affecting Cell Cycle. (A) Quantification of K-Ras mRNA (qRT-PCR) and protein by Western blot in hearts of ∝-MHC miR-1 transgenic mice (Tg) compared to wild type (Wt) littermates. (B) Quantification of Smyd3 (enriched>12 fold) mRNA (qRT-PCR) and protein by Western blot in hearts of miR-1 Tg compared to Wt littermates. Smyd3 was not detectable in HL-1 cells. (C) Quantification of Cdk6 mRNA (qRT-PCR) and protein by Western blot in hearts of miR-1 Tg compared to Wt littermates, or after knockdown (KD) of miR-1 in HL-1 cells. (D) Quantification of Ocrl (>16 fold enrichment) mRNA (qRT-PCR) and protein by Western blot in hearts of miR-1 Tg compared to Wt littermates. Representative Western blots are shown below each graph with GAPDH as loading control. Error bars represent standard deviations and asterisks indicate p<0.05.

FIG. 10. Putative miR-1 binding sites in miR-1 pull-down enriched targets. (A) Conserved putative binding site for miR-1 in the 3′UTR of K-ras containing hexameric 5′ base-pairing but additional compensatory base-pairing to nts 10-15 of miR-1. (B) Putative binding sites for miR-1 in the coding region and 3′UTR of mouse and human Smyd3. (C) Putative binding sties for miR-1 in the 3′UTR of mouse and human cdk6. (D) Conserved putative binding sties for miR-1 in the Ocrl 3′UTR.

Evidence for a Non-canonical miRNA Seed

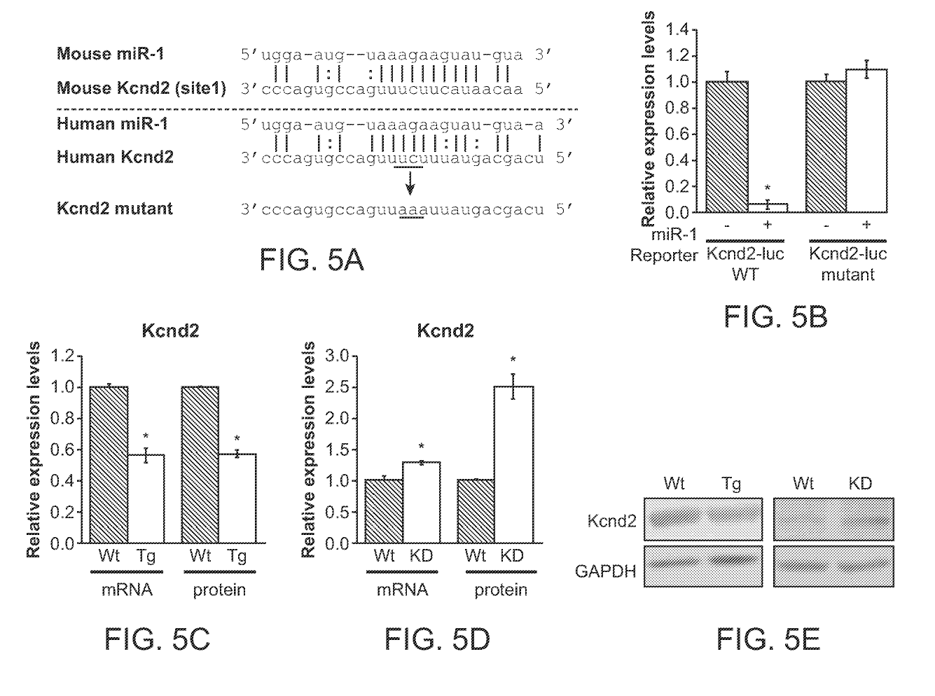

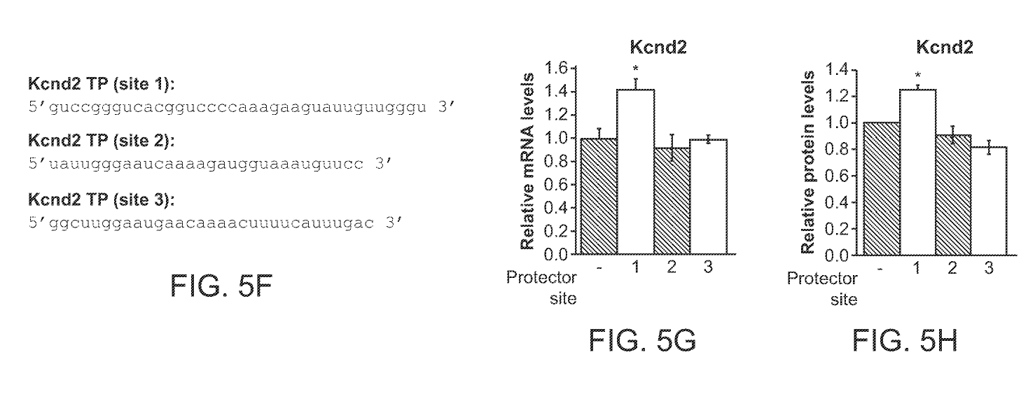

Of transcripts enriched ≧8-fold, ˜25% contained a sequence matching the mid-portion of miR-1 (heptamers complementary to the region of nts 9-17 with highest frequency for complementarity to bases 9-15 of miR-1, p<10⁻⁵) (FIG. 1C) but no 5′ seed match (FIG. 1E). Hence, we investigated whether this region (9-17) of miR-1, like the well-described 5′ seed, represses mRNAs in a sequence-dependent manner. We first focused on one of the most enriched transcripts from our screen, Kcnd2 (18-fold), a potassium channel that contributes to the transient outward channel and determines the timing of cardiac repolarization; disruption of this precisely coordinated process increases susceptibility to lethal arrhythmias (Costantini et al., 2005). Kcnd2 mRNA has a 5′ seed match, but this sequence did not mediate repression in luciferase assays (site 3, FIGS. S4A and S4B). However, a conserved region in the coding sequence had a match to miR-1 nts 8-18 but not to the 5′ seed (site 1, FIG. 5A).

To isolate the potential function of this unusual site and to exclude the possibility that an occult imperfect 5′ seed mediates miR-1-dependent repression, we generated concatamers of the putative miR-1 binding site that lacks any 5′ seed match. The site efficiently repressed luciferase activity in a heterologous reporter assay (FIG. 5B). Mutations in the binding site corresponding to bases 10-12 of miR-1 (FIG. 5A) made this sequence unresponsive to miR-1 (FIG. 5B). Accordingly, Kcnd2 protein and mRNA levels were downregulated in hearts overexpressing miR-1 (FIGS. 5C and 5E), while knockdown of miR-1 in HL-1 cells significantly increased Kcnd2 protein levels (FIGS. 5D and 5E). The significant up-regulation of Kcnd2 observed with knockdown of miR-1 is consistent with the high enrichment of Kcnd2 in our pull-down assay and severe repression in luciferase assays.

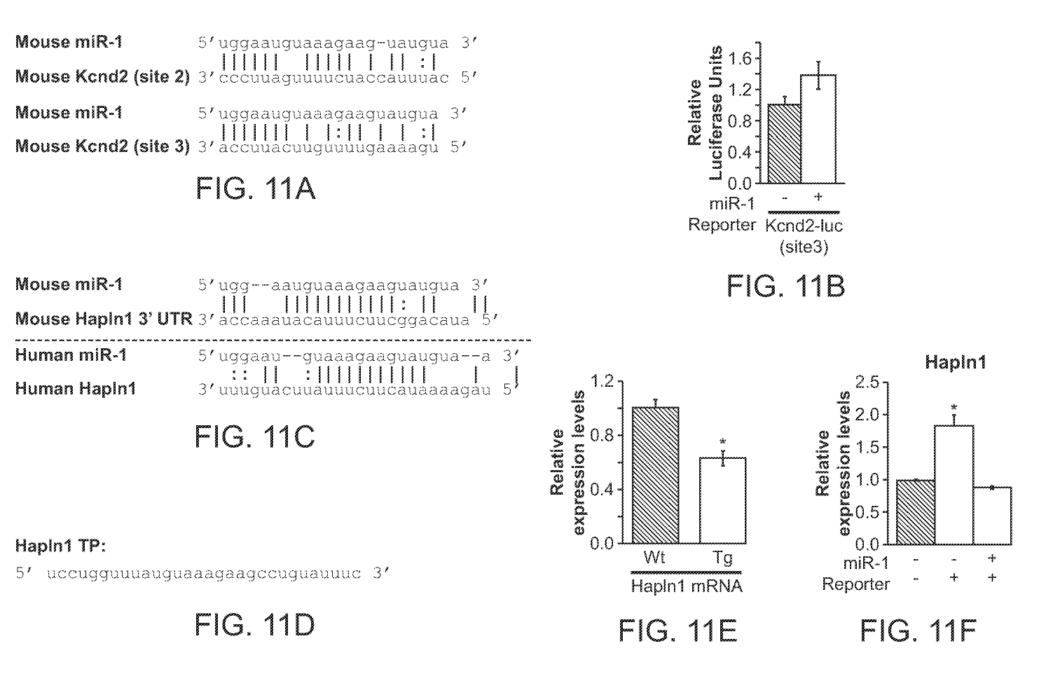

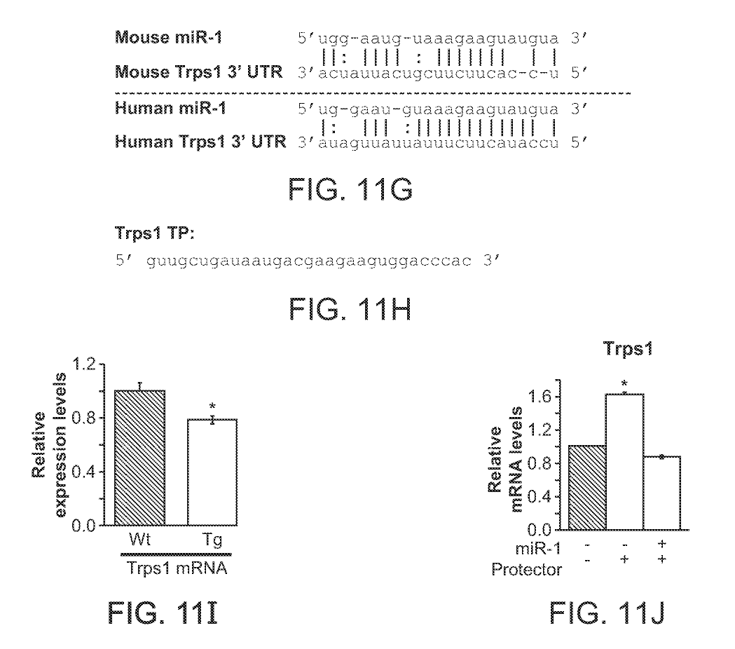

To determine if nucleotides 8-18 mediate repression by miR-1 in cardiomyocytes, we transfected HL-1 cells expressing Kcnd2 with a sequence complementary to the novel miR-1 binding site (site 1) in Kcnd2 using a technology known as target protection (Choi et al., 2007) (FIG. 5F). The “protector” is an oligonucleotide that competes with miR-1 to bind the Kcnd2 transcript and protects Kcnd2 from miR-1-mediated repression. As controls, target protectors complementary to the site containing a 5′ seed to miR-1 (site 3) (FIGS. 5F and 11A) that could not mediate repression in luciferase assays and to another site (site 2) in the Kcnd2 3′ UTR with modest base-pairing along the length of miR-1 were used (FIGS. 5F and 11A). Only the target protector for site 1 increased Kcnd2 mRNA and levels (FIGS. 5G and 5H), further supporting that the novel seed sequence supports miRNA-mediated repression in cells.

FIG. 5. Evidence for an Alternate Seed Sequence for miRNA-Mediated Repression. (A) Conserved sequence complementarity of the mid-region of miR-1 to Kcnd2 3′ UTR in mouse and human with lack of 5′ seed complementarity (site 1). The three nucleotide mutation in area of complementarity for studies in (B) is indicated. (B) Repression of luciferase activity by miR-1 upon insertion of binding site in (A) into luciferase 3′ UTR (Kcnd2-luc) that was abolished by mutation of core of binding site. (C) Quantification of Kcnd2 mRNA (qRT-PCR) and protein by Western blot in hearts of miR-1 expressing transgenic mice (Tg) compared to wild type (Wt) littermates shows repression. (D) Quantification of Kcnd2 mRNA (qRT-PCR) or protein by Western blot after knockdown (KD) of miR-1 in HL-1 cells shows upregulation of Kcnd2 protein. (E) Representative Western blots of Kcnd2 and GAPDH protein in Tg or Wt hearts. (F) Sequences for “target protectors” designed to occupy three different regions of Kcnd2 mRNA for potential inhibition of miR-1 function. (G, H) Target protectors designed to protect potential miR-1 sites 1, 2 or 3 in the Kcnd2 mRNA from miR-1 effects showed protection of Kcnd2 mRNA and protein levels only with site1 protector in HL-1 atrial cardiomyocyte cells. Error bars indicate standard deviation and asterisks indicate p<0.05.

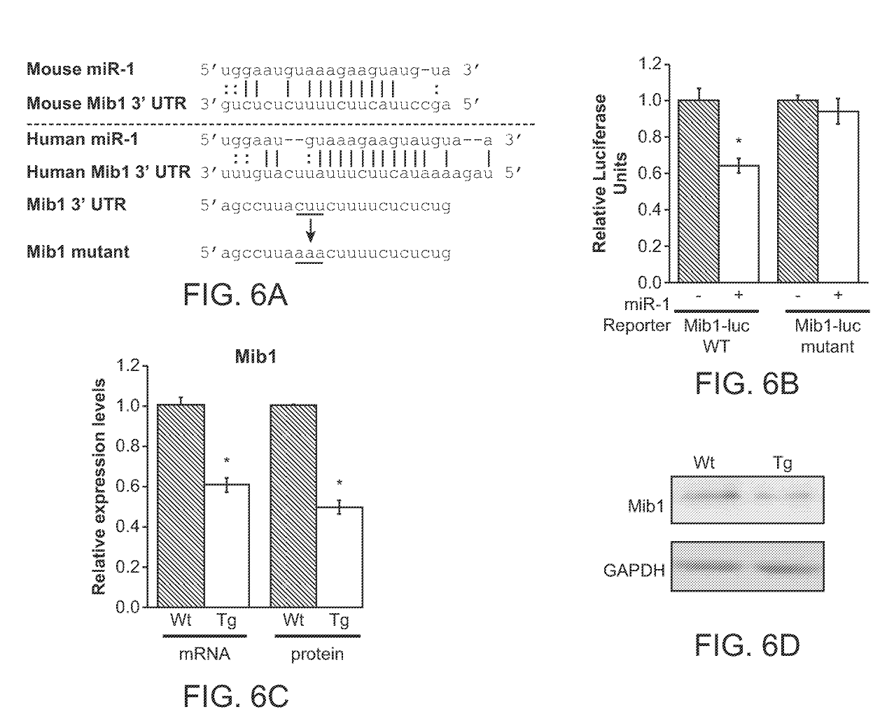

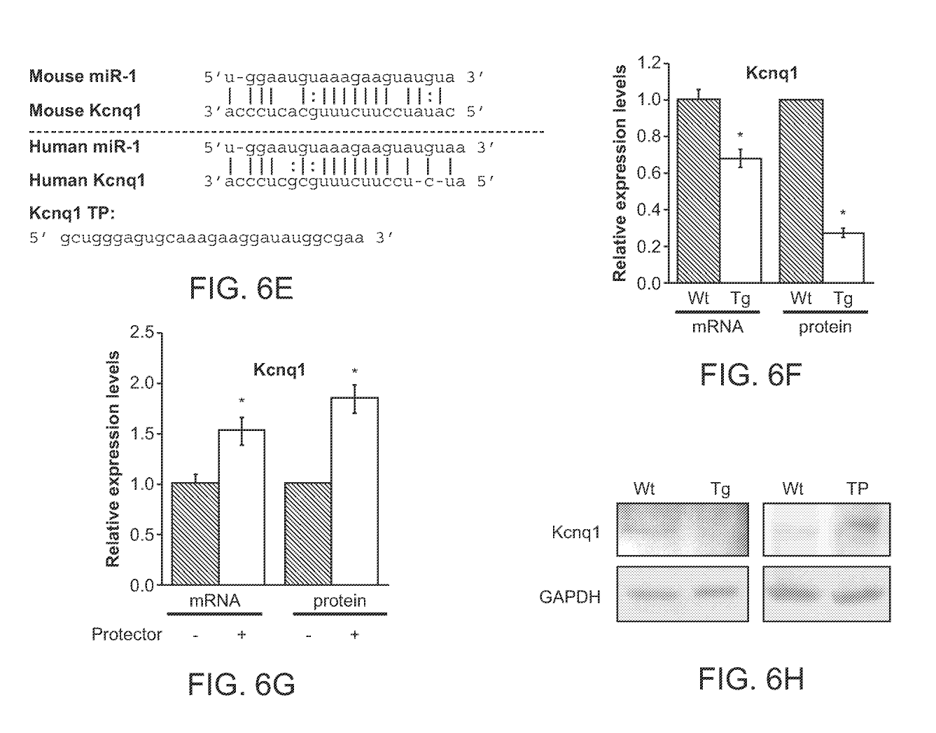

To determine if repression of Kcnd2 mediated by a non-canonical seed was an isolated event, we searched for other heptamer matches within nts 9-17 that might function as miRNA seeds. One such mRNA was Mindbomb1 (Mib1), enriched 11-fold in our screen, and also the gene within which the bicistronic miR-1-2 and miR-133-2 transcript resides (Zhao et al., 2007). Mib1 did not have an intact 5′ seed match in its annotated transcript but did have a conserved sequence complementary to nts 9-17 of miR-1 in the 3′ UTR (FIG. 6A). Upon introduction of miR-1, the non-canonical binding site containing a match to miR-1 nts 9-17 repressed luciferase activity (FIG. 6B). The repression was alleviated upon mutation of the binding site, corresponding to nts 13-15 of miR-1 (FIGS. 6A and 6B). Mib1 protein and mRNA levels were reduced in heart lysates from transgenic mice overexpressing miR-1 (FIGS. 6C and 6D). The regulation of Mib1 by miR-1 is consistent with the 1.4-fold up-regulation of Mib-1 mRNA in mice lacking miR-1-2 (Zhao et al., 2007).

The multiple lines of evidence, which are the current standard for validating miRNA-mediated repression of mRNA targets, indicate that miR-1 represses Kcnd2 and Mib1 through a sequence match with nts 9-17, even in the absence of 5′ seed complementarity. Thus, the novel seed sequence appears to be both necessary and sufficient for repressive activity.

We also investigated whether transcripts that contained the novel seed but were enriched <8-fold were regulated by miR-1. Kcnq1, a potassium channel protein often mutated in human cardiac arrhythmias (Wang et al., 1996), was enriched >3-fold in our biochemical pull-down assay. The coding region of Kcnq1 contains a sequence complementary to miR-1 nts 7-15 (FIG. 6E). Kcnq1 mRNA and protein levels were reduced in miR-1-overexpressing hearts (FIGS. 6F and 6H). Introduction of a target protector specific to the putative miR-1 binding site possessing the novel middle seed (FIG. 6E) increased Kcnq1 mRNA and protein levels (FIGS. 6G and 6H). Thus, miR-1 may mediate repression of Kcnq1 in part by binding to a sequence complementary to nts 7-15 of miR-1.

FIG. 6. Validation of the Alternate Seed Sequence for miR-1-Mediated Repression on Additional Targets. (A) Conserved sequence complementarity of the mid-region (nt 9-17) of miR-1 to Mindbomb1 (Mib1) 3′ UTR in mouse and human with lack of 5′ seed complementarity. The three nucleotide mutation in area of complementarity for studies in (B) is indicated. (B) Repression of luciferase activity by miR-1 upon insertion of tandem binding sites in (A) into luciferase 3′ UTR (Mib1-luc) that was abolished by mutation of core of binding site. (C) Quantification of Mib1 mRNA (qRT-PCR) and protein by Western blot in hearts of miR-1 expressing transgenic mice (Tg) compared to wild type (Wt) littermates shows repression. (D) Representative Western blot of Mib1 and GAPDH protein in Tg or Wt hearts. (E) Conserved sequence complementarity of the mid-region (nt 7-15) of miR-1 to Kcnq1 3′ UTR in mouse and human with lack of 5′ seed. Target protector sequence used for inhibiting miR-1 function on this site is shown. (F) Quantification of Kcnq1 mRNA (qRT-PCR) and protein by Western blot in hearts of miR-1 Tg compared to Wt littermates shows repression. (G) Quantification of Kcnq1 mRNA (qRT-PCR) and protein by Western blot with or without introduction of the target protector for inhibition of miR-1 function shows increased levels in HL-1 cells. (H) Representative Western blot of Kcnq1 and GAPDH in Tg or Wt hearts and in Wt or target protected (TP) HL-1 cardiomyocyte cells. Error bars indicate standard deviation and asterisks indicate p<0.05.

Finally, we investigated several other highly enriched transcripts that did not have canonical 5′ seed matches. We found that Hapln1/Crtl1 (Wirrig et al., 2007) and Trps1 (Savinainen et al., 2004) had complementary regions to nts 5-15 or 10-16, respectively, and we validated these by specific target protector assays and analysis of mRNA levels (FIGS. 11C-J). These findings suggest a broader significance for the middle region of miR-1 in sequence-dependent repression.

FIG. 11. Additional evidence for the novel seed region in miR-1-mediated repression. (A) Additional regions in Kcnd2 3′UTR with miR-1 complementarity including one with an incomplete 5′ seed (site 2) or a classic 5′ seed (site 3). (B) Site 3 in Kcnd2 3′UTR was unable to repress luciferase activity upon introduction of exogenous miR-1 despite the presence of a 5′ seed for miR-1. (C) Hapln1, enriched ˜10-fold in the biochemical screen, lacks an intact 5′ seed but possesses conserved base-pairing to the novel alternate middle seed region (nt 7-16) of miR-1. (D,E,F) Conserved sequence complementarity of nt 9-15 of miR-1 with Hpaln1 3UTR. Hapln1 mRNA levels were lower in α-MHC miR-1 expressing mice. Hapln1 mRNA in HL-1 cells was increased with a target protector complementary to the novel seed site; this effect was specifically reversed by excess miR-1. (G,H,I,J) Regulation of Trps by miR-1. Trps1 miRNA levels were modestly downregulated in myocardium of α-MHC miR-1-expressing mice. Trps1 mRNA levels were increased in HL-1 cells by using a target protector complementary to the novel seed site with base pairing to nts 10-16 of miR-1. This effect was reversed by addition of excess miR-1.

Electrophysiologic Abnormalities in α-MHC-miR-1 Transgenic Mice Correspond to miR-1 Targets

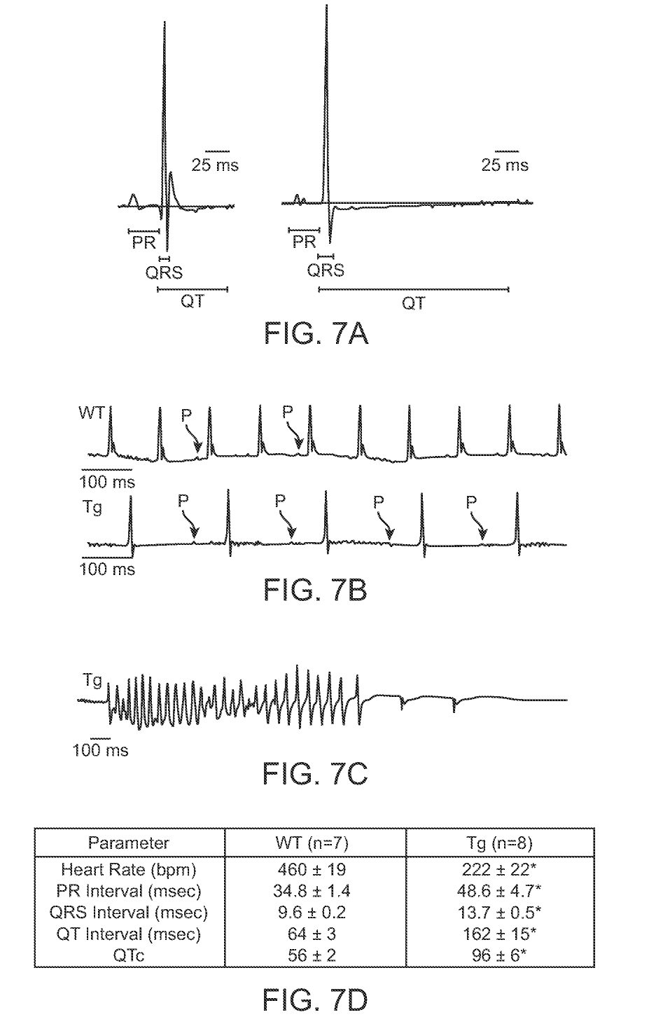

To determine if the numerous ion channels isolated from our screen were consistent with the in vivo functions of miR-1, we characterized the electrophysiology of α-MHC-miR-1 transgenic mice used in this study. The mice were grossly normal, but after 6 months of age, they began to manifest sudden death. Despite normal cardiac function, electrocardiography (ECG) revealed marked differences between wildtype and transgenic mice as early as 6 weeks of age (FIG. 7A). The transgenic mice had slower heart rates (FIG. 7B), prolonged atrial (PR interval) and ventricular (QRS interval) conduction times, and markedly prolonged ventricular repolarization times (QT interval) (FIGS. 7A and 7D). During ECG recordings, several mice had a fatal ventricular tachycardia typically caused by delayed ventricular repolarization in the setting of slow heart rate (FIG. 7C). The slow heart rate and delayed conduction are consistent with the notion that several of the miR-1 targets encode ion channels, providing in vivo physiologic validation of this category of targets.

FIG. 7. miR-1-2 Overexpression Affects Cardiac Electrophysiology. (A) Examples of a multilead surface electrocardiogram in an anesthetized adult ∝-MHC miR-1-2-overexpressing mouse (Tg) and a wild type (Wt) littermate. The transgenic mice have several abnormalities: the P-wave (arrow), representing atrial depolarization, is broadened with lower amplitude. The PR and QRS intervals, reflecting transit time between the atrium and ventricle and ventricular activation times respectively, are. The QT interval, corresponding to ventricular repolarization, was markedly prolonged also. (B) Example of sinus bradycardia (slow heart rate) observed in ∝-MHC miR-1-2-overexpressing transgenic mice. (C) Example of surface electrocardiogram showing ventricular arrhythmia (tachycardia) in anesthetized ∝-MHC miR-1-2-overexpressing transgenic mice. In humans, this abnormal rhythm, called torsades de pointes, is caused by repolarization abnormalities (e.g., in the long QT syndrome). (D) Quantification of electrocardiography measurements demonstrating abnormalities in cardiac conduction in transgenic animals. * indicates p<0.01.

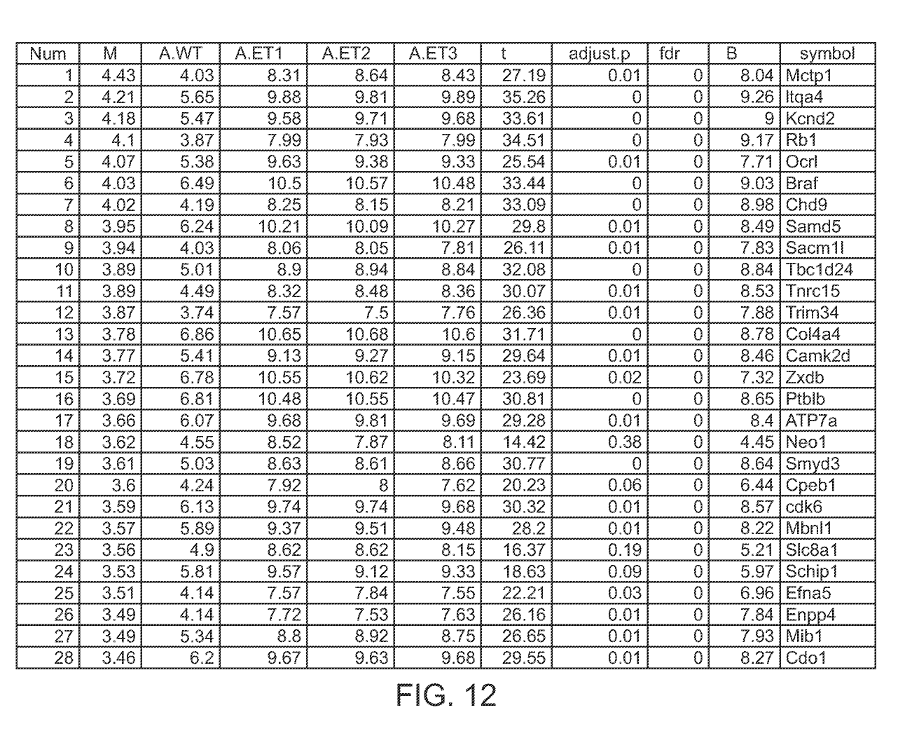

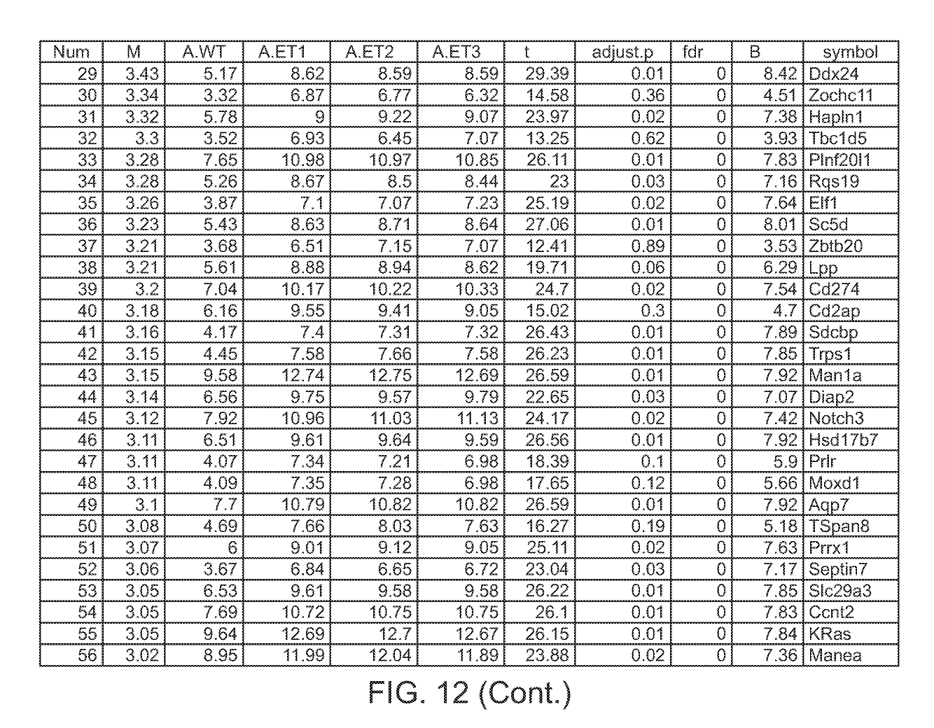

FIG. 12. List of annotated mRNAs enriched ≧8-fold in miR-1 pulldown assay. T=t-statistics testing if the mean of eluates is different from the mean of inputs. Fdr=False discovery rate p values. B=log-posterior odds of differential expression. B>0 means that a subset is more likely to be differentially expressed than not.

Repression Mediated by the Middle Region of miR-195

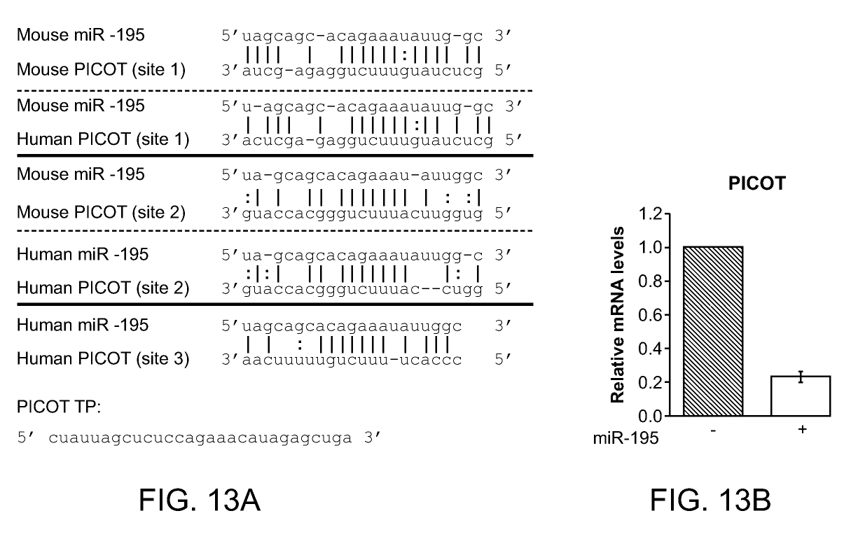

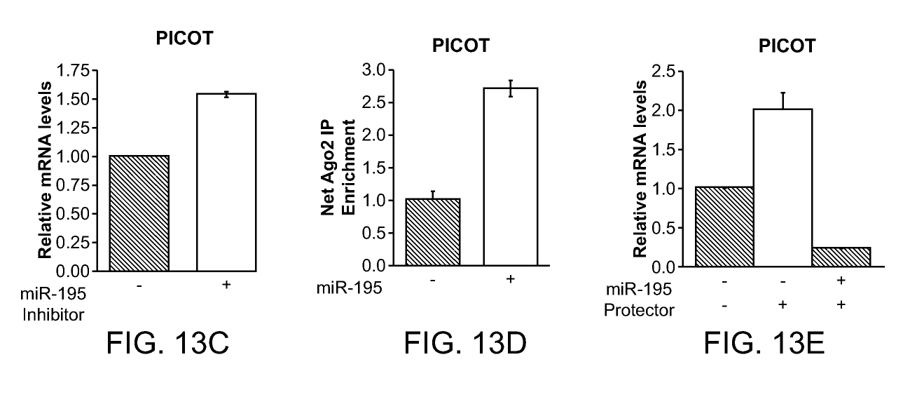

It was investigated whether repression mediated by the middle region of a miRNA was unique to miR-1 or might also be observed with other miRNAs. miR-195 can cause cardiac hypertrophy when overexpressed, but the targets are not known. Van Rooij et al. (2006) Proc. Natl. Acad. Sci. USA 103:18255. A search was conducted for mRNAs involved in hypertrophy that also had complementarity to the middle region of miR-195. It was found that the coding sequence of PICOT (PKC-Interacting Cousin of Thioredoxin) had a sequence match with nts 9-19 of miR-195 (FIG. 13A). PICOT functions as a negative regulator of hypertrophy by displacing the phosphatase Calcineurin, a facilitator of hypertrophy, from its docking site. Jeong et al. (2008) Circ. Res. 102:711. Depletion of PICOT would allow Calcineurin to remain anchored to the Z-disk of muscle where it promotes NFATc dephosphorylation and transport to the nucleus, resulting in hypertrophy. Jeong et al. (2008) supra. PICOT mRNA levels were severely downregulated upon transfection of miR-195 into HL-1 cells and upregulated upon addition of miR-195 inhibitor (FIGS. 13B and 13C). The decrease in mRNA levels correlated with enrichment of PICOT mRNA in a miR-195-dependent Ago2 pull-down (FIG. 13D). A target protector antisense to the non-canonical miR-195 binding site elevated PICOT mRNA levels in HL-1 cells, and this increase could be competed away with excess miR-195 (FIGS. 13A and 13E). These findings provide evidence that the alternate seed in PICOT harboring extensive base-pairing with the middle region of miR-195 was functional in cardiomyocytes.

FIGS. 13A-E. Repression mediated by the middle region of miR-195. (A) Conserved sequence complementarity of the mid-region of miR-195 to PICOT mRNA in coding region of mouse and human; note lack of 5′ seed complementarity. Sequence of a target protector corresponding to site 1 in PICOT is shown. (B,C) Decrease in PICOT mRNA levels upon addition of miR-195 and reciprocal increase in mRNA levels upon inhibition of endogenous miR-195 present in the HL-1 cardiomyocyte cell line. (D) The decrease in PICOT mRNA correlated with an increase in association of the transcript in a miR-195 dependent Argonaute2 pull-down. (E) The target protector introduced into HL-1 cells resulted in increased mRNA levels of PICOT. This rescue was alleviated using excess miR-195.

### REFERENCES

Backs, J., Song, K., Bezprozvannaya, S., Chang, S., and Olson, E. N. (2006). CaM kinase II selectively signals to histone deacetylase 4 during cardiomyocyte hypertrophy. J Clin Invest 116, 1853-1864.
Beitzinger, M., Peters, L., Zhu, J. Y., Kremmer, E., and Meister, G. (2007). Identification of human microRNA targets from isolated argonaute protein complexes. RNA Biol 4, 76-84.
Berman, D. M., Wilkie, T. M., and Gilman, A. G. (1996). GAIP and RGS4 are GTPase-activating proteins for the Gi subfamily of G protein alpha subunits. Cell 86, 445-452.
Bers, D. M. (2002). Cardiac excitation-contraction coupling. Nature 415, 198-205.
Bogdanov, K. Y., Vinogradova, T. M., and Lakatta, E. G. (2001). Sinoatrial nodal cell ryanodine receptor and Na(+)—Ca(2+) exchanger: molecular partners in pacemaker regulation. Circ Res 88, 1254-1258.
Chen, J. F., Mandel, E. M., Thomson, J. M., Wu, Q., Callis, T. E., Hammond, S. M., Conlon, F. L., and Wang, D. Z. (2006). The role of microRNA-1 and microRNA-133 in skeletal muscle proliferation and differentiation. Nat Genet 38, 228-233.
Choi, W. Y., Giraldez, A. J., and Schier, A. F. (2007). Target protectors reveal dampening and balancing of Nodal agonist and antagonist by miR-430. Science 318, 271-274.
Costantini, D. L., Arruda, E. P., Agarwal, P., Kim, K. H., Zhu, Y., Zhu, W., Lebel, M., Cheng, C. W., Park, C. Y., Pierce, S. A., et al. (2005). The homeodomain transcription factor Irx5 establishes the mouse cardiac ventricular repolarization gradient. Cell 123, 347-358.
Croce, C. M. (2008). Oncogenes and cancer. N Engl J Med 358, 502-511.
Didiano, D., and Hobert, O. (2006). Perfect seed pairing is not a generally reliable predictor for miRNA-target interactions. Nat Struct Mol Biol 13, 849-851.
Dinulescu, D. M., Ince, T. A., Quade, B. J., Shafer, S. A., Crowley, D., and Jacks, T. (2005). Role of K-ras and Pten in the development of mouse models of endometriosis and endometrioid ovarian cancer. Nat Med 11, 63-70.
Easow, G., Teleman, A. A., and Cohen, S. M. (2007). Isolation of microRNA targets by miRNP immunopurification. Rna 13, 1198-1204.
Filipowicz, W., Bhattacharyya, S. N., and Sonenberg, N. (2008). Mechanisms of post-transcriptional regulation by microRNAs: are the answers in sight? Nat Rev Genet 9, 102-114.
Grimson, A., Farh, K. K., Johnston, W. K., Garrett-Engele, P., Lim, L. P., and Bartel, D. P. (2007). MicroRNA targeting specificity in mammals: determinants beyond seed pairing. Mol Cell 27, 91-105.
Groisman, I., Huang, Y. S., Mendez, R., Cao, Q., Theurkauf, W., and Richter, J. D. (2000). CPEB, maskin, and cyclin B1 mRNA at the mitotic apparatus: implications for local translational control of cell division. Cell 103, 435-447.
Haigis, K. M., Kendall, K. R., Wang, Y., Cheung, A., Haigis, M. C., Glickman, J. N., Niwa-Kawakita, M., Sweet-Cordero, A., Sebolt-Leopold, J., Shannon, K. M., et al. (2008). Differential effects of oncogenic K-Ras and N-Ras on proliferation, differentiation and tumor progression in the colon. Nat Genet.
Hamamoto, R., Furukawa, Y., Morita, M., Iimura, Y., Silva, F. P., Li, M., Yagyu, R., and Nakamura, Y. (2004). SMYD3 encodes a histone methyltransferase involved in the proliferation of cancer cells. Nat Cell Biol 6, 731-740.
Ivey, K. N., Muth, A., Arnold, J., King, F. W., Yeh, R. F., Fish, J. E., Hsiao, E. C., Schwartz, R. J., Conklin, B. R., Bernstein, H. S., and Srivastava, D. (2008). MicroRNA regulation of cell lineages in mouse and human embryonic stem cells. Cell Stem Cell 2, 219-229.
Johnson, L., Mercer, K., Greenbaum, D., Bronson, R. T., Crowley, D., Tuveson, D. A., and Jacks, T. (2001). Somatic activation of the K-ras oncogene causes early onset lung cancer in mice. Nature 410, 1111-1116.
Karginov, F. V., Conaco, C., Xuan, Z., Schmidt, B. H., Parker, J. S., Mandel, G., and Hannon, G. J. (2007). A biochemical approach to identifying microRNA targets. Proc Natl Acad Sci U S A 104, 19291-19296.
Kumar, M. S., Lu, J., Mercer, K. L., Golub, T. R., and Jacks, T. (2007). Impaired microRNA processing enhances cellular transformation and tumorigenesis. Nat Genet 39, 673-677.
Kwon, C., Han, Z., Olson, E. N., and Srivastava, D. (2005). MicroRNA1 influences cardiac differentiation in Drosophila and regulates Notch signaling. Proc Natl Acad Sci U S A 102, 18986-18991.
Long, D., Lee, R., Williams, P., Chan, C. Y., Ambros, V., and Ding, Y. (2007). Potent effect of target structure on microRNA function. Nat Struct Mol Biol 14, 287-294.
Malumbres, M., and Barbacid, M. (2005). Mammalian cyclin-dependent kinases. Trends Biochem Sci 30, 630-641.
Mitchell, G. F., Jeron, A., and Koren, G. (1998). Measurement of heart rate and Q-T interval in the conscious mouse. Am J Physiol 274, H747-751.
Pandit, B., Sarkozy, A., Pennacchio, L. A., Carta, C., Oishi, K., Martinelli, S., Pogna, E. A., Schackwitz, W., Ustaszewska, A., Landstrom, A., et al. (2007). Gain-of-function RAF1 mutations cause Noonan and LEOPARD syndromes with hypertrophic cardiomyopathy. Nat Genet 39, 1007-1012.
Richter, J. D. (2007). CPEB: a life in translation. Trends Biochem Sci 32, 279-285.
Savinainen, K. J., Linja, M. J., Saramaki, O. R., Tammela, T. L., Chang, G. T., Brinkmann, A. O., and Visakorpi, T. (2004). Expression and copy number analysis of TRPS1, EIF3S3 and MYC genes in breast and prostate cancer. Br J Cancer 90, 1041-1046.
Sethupathy, P., Megraw, M., and Hatzigeorgiou, A. G. (2006). A guide through present computational approaches for the identification of mammalian microRNA targets. Nat Methods 3, 881-886.
Sokol, N. S., and Ambros, V. (2005). Mesodermally expressed Drosophila microRNA-1 is regulated by Twist and is required in muscles during larval growth. Genes Dev 19, 2343-2354.
Stefani, G., and Slack, F. (2006). MicroRNAs in search of a target. Cold Spring Harb Symp Quant Biol 71, 129-134.
Stefani, G., and Slack, F. J. (2008). Small non-coding RNAs in animal development. Nat Rev Mol Cell Biol 9, 219-230.
Vatolin, S., Navaratne, K., and Weil, R. J. (2006). A novel method to detect functional microRNA targets. J Mol Biol 358, 983-996.
Vinogradova, T. M., Zhou, Y. Y., Bogdanov, K. Y., Yang, D., Kuschel, M., Cheng, H., and Xiao, R. P. (2000). Sinoatrial node pacemaker activity requires Ca(2+)/calmodulin-dependent protein kinase II activation. Circ Res 87, 760-767.
Vinther, J., Hedegaard, M. M., Gardner, P. P., Andersen, J. S., and Arctander, P. (2006). Identification of miRNA targets with stable isotope labeling by amino acids in cell culture. Nucleic Acids Res 34, e107.
Wang, Q., Curran, M. E., Splawski, I., Burn, T. C., Millholland, J. M., VanRaay, T. J., Shen, J., Timothy, K. W., Vincent, G. M., de Jager, T., et al. (1996). Positional cloning of a novel potassium channel gene: KVLQT1 mutations cause cardiac arrhythmias. Nat Genet 12, 17-23.
Wirrig, E. E., Snarr, B. S., Chintalapudi, M. R., O'Neal J, L., Phelps, A. L., Barth, J. L., Fresco, V. M., Kern, C. B., Mjaatvedt, C. H., Toole, B. P., et al. (2007). Cartilage link protein 1 (Crtl1), an extracellular matrix component playing an important role in heart development. Dev Biol 310, 291-303.
Yang, B., Lin, H., Xiao, J., Lu, Y., Luo, X., Li, B., Zhang, Y., Xu, C., Bai, Y., Wang, H., et al. (2007). The muscle-specific microRNA miR-1 regulates cardiac arrhythmogenic potential by targeting GJA1 and KCNJ2. Nat Med 13, 486-491.
Zhao, Y., Ransom, J. F., Li, A., Vedantham, V., von Drehle, M., Muth, A. N., Tsuchihashi, T., McManus, M. T., Schwartz, R. J., and Srivastava, D. (2007). Dysregulation of cardiogenesis, cardiac conduction, and cell cycle in mice lacking miRNA-1-2. Cell 129, 303-317.
Zhao, Y., Samal, E., and Srivastava, D. (2005). Serum response factor regulates a muscle-specific microRNA that targets Hand2 during cardiogenesis. Nature 436, 214-220.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of identifying a target of a microRNA (miRNA), the method comprising:
a) contacting the miRNA with a plurality of mRNA under conditions that favor duplex formation between the miRNA and at least one member of the plurality of mRNA,
b) eluting any mRNA that forms a duplex with the miRNA in step (a).

2. The method of claim 1, wherein the miRNA is immobilized on a solid support.

3. The method of claim 1, wherein the miRNA is immobilized via its 3′ terminus.

4. The method of claim 2, wherein the solid support comprises a specific binding partner for a binding moiety on the miRNA.

5. The method of claim 4, wherein the 3′ terminal nucleotide of the miRNA is modified with an amine group, and wherein the amine-modified miRNA is biotinylated.

6. The method of claim 5, wherein the specific binding partner is streptavidin.

7. The method of claim 1, wherein the target plurality of mRNA comprises mRNA lacking a canonical 5′ seed sequence at bases 1-7, 2-7, or 2-8.

8. The method of claim 1, further comprising synthesizing cDNA using the eluted mRNA as template.

9. The method of claim 8, further comprising sequencing the cDNA.

10. The method of claim 8, further comprising contacting the cDNA with an array of nucleic acid probes of known sequence under conditions that favor hybridization of the cDNA with at least one member of the probe array.

11. The method of claim 10, wherein the cDNA comprises a detectable label.

12. The method of claim 10, wherein hybridization of the cDNA to the at least one member of the probe array provides information as to the identity of the cDNA.

13. The method of claim 1, wherein the plurality of mRNA is isolated from a stem cell, a differentiated cell, or a cell that has been exposed to a stimulus.

14. The method of claim 13, wherein the plurality of mRNA is isolated from a cell that has been exposed to a stimulus, and wherein the stimulus is contact with an infectious agent, change in pH of cell culture medium, change in temperature, electrical charge, change in ion concentration of cell culture medium, contact with an effector molecule, or genetic modification.

15. The method of claim 1, wherein the plurality of mRNA is isolated from a diseased tissue.

16. The method of claim 1, wherein the plurality of mRNA is isolated from a non-diseased tissue.

17. The method of claim 15, wherein the plurality of mRNA is isolated from a tumor cell.

18. The method of claim 1, wherein the plurality of mRNA is isolated from a cell selected from a myoblast, a neutrophil, an osteoblast, a chondrocyte, a basophil, an eosinophil, an adipocyte, a neuron, a glial cell, a melanocyte, an epithelial cell, an endothelial cell, a stem cell, and a cell that has been selected or sorted on the basis of expression of a cell surface protein.

19. The method of claim 1, wherein the plurality of mRNA is pre-selected, to generate a sub-population of mRNA, and wherein the plurality of mRNA comprises the sub-population of mRNA.

20. The method of claim 19, wherein the pre-selection is on the basis of size, sequence, or polyadenylation.
